SEQUENCE LISTING (2 sequences)

SEQ ID NO: 1 - protein, 9 aa, Artificial Sequence
  Synthetic Construct
XXXXENGXX

SEQ ID NO: 2 - protein, 18 aa, Artificial Sequence
  Synthetic Construct
XXXXXXXXXGXXXXDXXX

(19) United States
(12) Patent Application Publication
(10) Pub. No.: US 2014/0342418 A1
(43) Pub. Date: Nov. 20, 2014

(54) XYLITOL PRODUCTION FROM CELLULOSIC BIOMASS

(71) Applicants: THE BOARD OF TRUSTEES OFTHE UNIVERSITY OF ILLINOIS, Urbana, IL (US); THE REGENTS OF THE UNIVERSITY OF CALIFORNIA, Oakland, CA (US)

(72) Inventors: Yong-Su Jin, Champaign, IL (US); Suk-Jin Ha, Savoy, IL (US); Eun Joong Oh, Urbana, IL (US); Soo Rin Kim, Savoy, IL (US); Won Heong Lee, Champaign, IL (US); James H. Doudna Cate, Berkeley, CA (US); Jonathan M. Galazka, Berkeley, CA (US)

(21) Appl. No.: 14/350,285

(22) PCT Filed: Oct. 17, 2012
(86) PCT No.: PCT/US2012/060621

§ 371 Date: Apr. 7, 2014

Related U.S. Application Data
(60) Provisional application No. 61/548,191, filed on Oct. 17, 2011.

(51) Int. Cl. C12P 7/18

(52) U.S. Cl. CPC C12P 7/18
USPC 435/158

(57) ABSTRACT

The present disclosure relates to host cells containing a recombinant xylose reductase, a recombinant cellodextrin transporter, a recombinant intracellular β-glucosidase, and lacking xylitol dehydrogenase and xylulokinase, and to methods of using such cells for producing xylitol from cellulosic biomass containing cellodextrin and xylose.

### CROSS-REFERENCE TO RELATED APPLICATIONS

This application claims the benefit of U.S. Provisional Application No. 61/548,191, filed on Oct. 17, 2011, which is hereby incorporated by reference in its entirety.

### FIELD

The present disclosure relates to methods and compositions for producing xylitol from cellulosic biomass containing cellodextrin and xylose.

### BACKGROUND

Xylitol is a five-carbon sugar alcohol which has similar sweetness as sucrose (Hyvonen et al., 1982, Advances in food research, 28, 373-403). Xylitol has been widely used as a sugar substitute in various food products, such as chewing gum and candies, as xylitol not only inhibits dental caries but also has low calories (Granstrom et al., 2007, Applied microbiology and biotechnology, 74, 273-281; and Makinen, 1992, J Appl Nutr, 44, 16-28). In addition, xylitol is a high-value bio-based chemical that can be produced from sugars, as it can be used as a building block for various chemical compounds such as xylaric acid and glycols (Werpy, 2004, Top Value Added Chemicals From Biomass. Volume 1-Results of Screening for Potential Candidates From Sugars and Synthesis Gas. DTIC Document). While xylitol can be produced by a chemical hydrogenation process (Wisniak et al., 1974, Industrial & Engineering Chemistry Product Research and Development, 13, 75-79), the xylitol yields from such processes are lower than those from biological processes because of byproduct formation (U.S. Pat. No. 4,008,285). Thus, microbial production of xylitol has been studied as an alternative to the chemical production of xylitol (Nigam and Singh, 1995, Process Biochemistry, 30, 117-124).

Candida sp., a natural xylose-fermenting yeast, can convert xylose into xylitol. However, this yeast also uses xylose as a carbon source for cell growth and metabolism, which results in reduced xylitol yields (Chung et al., 2002, Enzyme and microbial technology, 30, 809-816). In addition, some Candida sp. show a pathogenic nature in opportunistic situations so that microbial production of xylitol by Candida sp. may be limited for large-scale fermentation (Granstrom et al., 2007, Applied microbiology and biotechnology, 74, 277-281). In this context, xylitol production by Saccharomyces cerevisiae may be advantageous for large-scale fermentation, because S. cerevisiae is generally recognized as safe (GRAS).

Although S. cerevisiae cannot ferment xylose, S. cerevisiae can be engineered to produce xylitol, as genetic manipulation of this yeast is easier than any other eukaryote (Romanos et al., 1992 Yeast, 8, 423-488). S. cerevisiae can be engineered to produce xylitol from xylose by introducing a xylose reductase gene from Scheffersomyces (Pichia) stipitis (XYL1) into the yeast. While engineered S. cerevisiae expressing XYL1 is expected to produce xylitol with a theoretical yield because the engineered strain cannot assimilate xylose as a carbon source,

co-substrates enabling cell growth and metabolism are required for xylitol production (Hallborn et al., 1991, Nature Biotechnology, 9, 1090-1095). Glucose can serve as such a co-substrate.

However, one problem with the use of glucose as a co-substrate is that glucose represses xylose transport (Hallborn et al., 1994, Applied microbiology and biotechnology, 42, 326-333; and Meinander and Hahn-Hagerdal, 1997, Biotechnol Bioeng, 54, 391-9). Xylose transport in S. cerevisiae is mediated by hexose transporters and the transportation of xylose by hexose transport is highly inhibited by glucose. Lee, W. J., Ryu, Y. W., Seo, J. H., 2000. Characterization of two-substrate fermentation processes for xylitol production using recombinant Saccharomyces cerevisiae containing xylose reductase gene. Process Biochemistry, 35, 1199-1203. Therefore, xylose can be converted into xylitol only after glucose depletion in a batch fermentation using a mixture of glucose and xylose (Trumbly, 1992, Molecular microbiology, 6, 15-21). Glucose-limited fed-batch fermentation can be performed to maintain low glucose concentration levels for efficient transportation of xylose into cells. Lee, W. J., Ryu, Y. W., Seo, J. H., 2000. Characterization of two-substrate fermentation processes for xylitol production using recombinant Saccharomyces cerevisiae containing xylose reductase gene. Process Biochemistry, 35, 1199-1203. However, controlling glucose concentrations at desired levels in a large-scale fermentation is not only difficult, but low levels of glucose can result in an insufficient supply of NADPH, which is used as a cofactor by the xylose reductase. Although other carbon sources, such as ethanol or glycerol, can be used as a co-substrate without inhibiting xylose transport, the cofactor regeneration capacities of ethanol and glycerol, under oxygen-limited conditions, are not as good as glucose. Due to these major problems with glucose, S. cerevisiae engineered to express a xylose reductase suffer from low volumetric productivity of xylitol, as compared to that the much higher xylitol yields produced by Candida sp.

One solution is to utilize cellodextrins, such as cellobiose, rather than glucose as a co-substrate. U.S. Patent Application Publication No. US 2011/0020910 and Ha et al., Proc Natl Acad Sci USA. 2011 Jan. 11; 108(2):504-9. Epub 2010 Dec. 27, disclose a S. cerevisiae strain engineered to express a cellodextrin transporter, a β-glucosidase, and a xylose metabolic pathway containing a xylose reductase, a xylitol dehydrogenase and a xylulokinase to produce ethanol by co-fermenting cellobiose and xylose. The xylose metabolic pathway converts xylose to phosphorylated xylulose, preventing xylitol accumulation in the cell and enhancing ethanol production. As U.S. Patent Application Publication No. US 2011/0020910 and Ha et al. relate to enhancing production of ethanol, neither publication discloses the production of xylitol by engineering a S. cerevisiae strain that only contains a xylose reductase in combination with a cellodextrin transporter and a β-glucosidase.

Accordingly, a need exists for improved engineered yeast strains that produce xylitol by utilizing a co-substrate that does not inhibit xylose transport and that regenerates sufficient amounts of cofactors during xylose conversion.

### BRIEF SUMMARY

In order to meet the above needs, the present disclosure provides methods of producing xylitol from cellulosic biomass containing cellodextrin and xylose and host cells containing a recombinant xylose reductase, a recombinant cellodextrin transporter, a recombinant intracellular β-glucosidase, and lacking xylitol dehydrogenase and xylulokinase.

Accordingly, certain aspects of the present disclosure relate to a method of producing xylitol, including: a) providing a host cell lacking xylitol deydrogenase and xylulokinase, where the host cell includes a recombinant xylose reductase, a recombinant cellodextrin transporter, and a recombinant β-glucosidase, and b) culturing the host cell in a medium including cellodextrin and xylose, whereby the host cell produces xylitol from the xylose. In certain embodiments, the recombinant xylose reductase may be XYL1 or GRE3.

In certain embodiments that may be combined with any of the preceding embodiments, the recombinant cellodextrin transporter may be a cellobiose transporter. In certain embodiments that may be combined with any of the preceding embodiments, the cellobiose transporter may be CDT-1. In certain embodiments that may be combined with any of the preceding embodiments, the recombinant β-glucosidase may be an intracellular β-glucosidase. In certain embodiments that may be combined with any of the preceding embodiments, the recombinant β-glucosidase may be GH1-1.

In certain embodiments that may be combined with any of the preceding embodiments, the host cell may further have reduced expression of a phosphoglucose isomerase. In certain embodiments, the phosphoglucose isomerase may be PGI1. In certain embodiments that may be combined with any of the preceding embodiments, the host cell may further include one or more recombinant polypeptides such as a glucose 6-phosphate dehydrogenase, a phosphoglucose isomerase, an acetyl-CoA synthetase, an isocitrate dehydrogenase, and an acetaldehyde dehydrogenase. In certain embodiments, expression of one or more recombinant polypeptides including a glucose 6-phosphate dehydrogenase, a phosphoglucose isomerase, an acetyl-CoA synthetase, an isocitrate dehydrogenase, and/or an acetaldehyde dehydrogenase results in an increase in the intracellular NADPH concentration in the host cell as compared to a corresponding cell. In certain embodiments, expression of one or more recombinant polypeptides including a glucose 6-phosphate dehydrogenase, a phosphoglucose isomerase, an acetyl-CoA synthetase, an isocitrate dehydrogenase, and/or an acetaldehyde dehydrogenase results in an increase in xylitol productivity in the host cell as compared to a corresponding cell. In certain embodiments, the glucose 6-phosphate dehydrogenase may be ZWF1. In certain embodiments, the phosphoglucose isomerase may be PGI1. In certain embodiments, the acetyl-CoA synthetase may be ACS1. In certain embodiments, the isocitrate dehydrogenase may be IDP2. In certain embodiments, the acetaldehyde dehydrogenase may be ALD6.

In certain embodiments that may be combined with any of the preceding embodiments, the growth medium may include cellodextrin and xylose in a g/L ratio of 1:1, 1:2, or 1:3. In certain embodiments that may be combined with any of the preceding embodiments, the host cell is cultured at a pH of at least 5, at least 5.5, at least 6, at least 6.5, or at least 7. In certain embodiments that may be combined with any of the preceding embodiments, the host cell may be a fungal cell. In certain embodiments that may be combined with any of the preceding embodiment, the host cell may be a yeast cell. In certain embodiments, the yeast cell may be S. cerevisiae.

Other aspects of the present disclosure relate to a host cell containing a recombinant xylose reductase, a recombinant cellodextrin transporter, and a recombinant intracellular β-glucosidase, where the host cell lacks xylitol dehydrogenase and xylulokinase. In certain embodiments, the recombinant xylose reductase may be XYL1 or GRE3. In certain embodiments, the recombinant xylose reductase may be GRE3. In certain embodiments, the recombinant xylose reductase may be XYL1. In certain embodiments that may be combined with any of the preceding embodiments, the recombinant cellodextrin transporter may be a cellobiose transporter. In certain embodiments, the cellobiose transporter may be CDT-1. In certain embodiments that may be combined with any of the preceding embodiments, the recombinant intracellular β-glucosidase may be GH1-1.

In certain embodiments that may be combined with any of the preceding embodiments, the host cell may further have reduced expression of a phosphoglucose isomerase. In certain embodiments, the phosphoglucose isomerase may be PGI1. In certain embodiments that may be combined with any of the preceding embodiments, the host cell may further include one or more recombinant polypeptides such as a glucose 6-phosphate dehydrogenase, a phosphoglucose isomerase, an acetyl-CoA synthetase, and isocitrate dehydrogenase, and an acetaldehyde dehydrogenase. In certain embodiments, expression of one or more recombinant polypeptides including a glucose 6-phosphate dehydrogenase, a phosphoglucose isomerase, an acetyl-CoA synthetase, an isocitrate dehydrogenase, and/or an acetaldehyde dehydrogenase results in an increase in the intracellular NADPH concentration in the host cell as compared to a corresponding cell. In certain embodiments, expression of one or more recombinant polypeptides including a glucose 6-phosphate dehydrogenase, a phosphoglucose isomerase, an acetyl-CoA synthetase, an isocitrate dehydrogenase, and/or an acetaldehyde dehydrogenase results in an increase in xylitol productivity in the host cell as compared to a corresponding cell. In certain embodiments, the glucose 6-phosphate dehydrogenase may be ZWF1. In certain embodiments, the phosphoglucose isomerase may be PGI1. In certain embodiments, the acetyl-CoA synthetase may be ACS 1. In certain embodiments, the isocitrate dehydrogenase may be IDP2. In certain embodiments, the acetaldehyde dehydrogenase may be ALD6. In certain embodiments that may be combined with any of the preceding embodiments, the host cell may be a fungal cell. In certain embodiments that may be combined with any of the preceding embodiments, the host cell may be a yeast cell. In certain embodiments, the yeast cell may be S. cerevisiae.

### DETAILED DESCRIPTION

### Overview

The present disclosure relates to host cells containing a recombinant xylose reductase, a recombinant cellodextrin transporter, and a recombinant intracellular β-glucosidase, where the host cell lacks xylitol dehydrogenase and xylulokinase. Further aspects of the present disclosure relate to methods of producing xylitol, by providing a host cell lacking xylitol deydrogenase and xylulokinase where said host cell includes a recombinant xylose reductase, a recombinant cellodextrin transporter, and a recombinant β-glucosidase; and culturing the host cell in a medium comprising cellodextrin and xylose whereby the host cell produces xylitol from the xylose. Moreover, the present disclosure is based, at least in part, on a novel strategy for producing xylitol from xylose via simultaneous co-fermentation of cellobiose and xylose, which are prevalent in cellulosic hydrolyzates, by utilizing an S. cerevisiae strain that was engineered to express the S. stipitis xylose reductase gene XYL1, the Neurospora crassa cellodextrin transporter gene cdt-1, and the Neurospora crassa intracellular β-glucosidase gene ghl-1. Advantageously, this engineered S. cerevisiae strain not only produced xylitol constitutively without glucose repression, but also showed higher xylitol production rates when cellobiose and xylose were co-utilized as compared to when glucose and xylose were sequentially utilized. Further, overexpression of NADP+ dependent dehydrogenases in this engineered strain advantageously increased intracellular NADPH concentrations and improved xylitol productivity.

### DEFINITIONS

Unless defined otherwise, all scientific and technical terms are understood to have the same meaning as commonly used in the art to which they pertain. For the purpose of the present disclosure, the following terms are defined.

As used herein, “cellodextrin” refers to glucose polymers of varying length and includes, without limitation, cellobiose (2 glucose monomers), cellotriose (3 glucose monomers), cellotetraose (4 glucose monomers), cellopentaose (5 glucose monomers), and cellohexaose (6 glucose monomers).

As used herein, a “cellodextrin transporter” refers to any sugar transport protein capable of transporting cellodextrins across the cell membrane of a cell.

As used herein, “sugar” refers to monosaccharides (e.g., glucose, fructose, galactose, xylose, arabinose), disaccharides (e.g., cellobiose, sucrose, lactose, maltose), and oligosaccharides (typically containing 3 to 10 component monosaccharides).

As used herein, the terms “polynucleotide,” “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; inter-nucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

As used herein, a “polypeptide” is an amino acid sequence containing a plurality of consecutive polymerized amino acid residues (e.g., at least about 15 consecutive polymerized amino acid residues, optionally at least about 30 consecutive polymerized amino acid residues, at least about 50 consecutive polymerized amino acid residues). In many instances, a polypeptide contains a polymerized amino acid residue sequence that is a transporter, an enzyme, a predicted protein of unknown function, or a domain or portion or fragment thereof. A transporter is involved in the movement of ions, small molecules, or macromolecules, such as a carbohydrate, across a biological membrane. An enzyme can catalyze a chemical reaction, such as the reduction of a carbohydrate to an alcohol, in a host cell. The polypeptide optionally contains modified amino acid residues, naturally occurring amino acid residues not encoded by a codon, and non-naturally occurring amino acid residues.

As used herein, “protein” refers to an amino acid sequence, oligopeptide, peptide, polypeptide, or portions thereof whether naturally occurring or synthetic.

Genes and proteins that may be used in the present disclosure include genes encoding conservatively modified variants and proteins that are conservatively modified variants of those genes and proteins described throughout the application. “Conservatively modified variants” as used herein include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Homologs of the genes and proteins described herein may also be used in the present disclosure. As used herein, “homology” refers to sequence similarity between a reference sequence and at least a fragment of a second sequence. Homologs may be identified by any method known in the art, preferably, by using the BLAST tool to compare a reference sequence to a single second sequence or fragment of a sequence or to a database of sequences. As described below, BLAST will compare sequences based upon percent identity and similarity. As used herein, “orthology” refers to genes in different species that derive from a common ancestor gene.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 29% identity, optionally 30%, 40%, 45%, 50%, 55%, 60%, 65%, 70%, 75%, 80%, 85%, 90%, 95%, 99% or 100% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200, or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1.

A “comparison window,” as used herein, includes reference to a segment of any one of the number of contiguous positions including, but not limited to from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1981), by the homology alignment algorithm of Needleman and Wunsch (1970) J Mol Biol 48(3):443-453, by the search for similarity method of Pearson and Lipman (1988) Proc Natl Acad Sci USA 85(8):2444-2448, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection [see, e.g., Brent et al., (2003) Current Protocols in Molecular Biology, John Wiley & Sons, Inc. (Ringbou Ed)].

Two examples of algorithms that are suitable for determining percent sequence identity and sequence similarity are the BLAST and BLAST 2.0 algorithms, which are described in Altschul et al. (1997) Nucleic Acids Res 25(17):3389-3402 and Altschul et al. (1990) J. Mol. Biol 215(3)-403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4, and a comparison of both strands. For amino acid sequences, the BLASTP program uses as defaults a word length of 3, and expectation (E) of 10, and the BLOSUM62 scoring matrix [see Henikoff and Henikoff, (1992) Proc Natl Acad Sci USA 89(22):10915-10919] alignments (B) of 50, expectation (E) of 10, M=5, N=−4, and a comparison of both strands.

The BLAST algorithm also performs a statistical analysis of the similarity between two sequences (see, e.g., Karlin and Altschul, (1993) Proc Natl Acad Sci USA 90(12):5873-5877). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Other than percentage of sequence identity noted above, another indication that two nucleic acid sequences or polypeptides are substantially identical is that the polypeptide encoded by the first nucleic acid is immunologically cross-reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

### Host Cells of the Present Disclosure

Certain aspects of the present disclosure relate to host cells containing a recombinant xylose reductase, a recombinant cellodextrin transporter, and a recombinant intracellular β-glucosidase, where the host cell lacks xylitol dehydrogenase and xylulokinase. Such host cells may be used to produce xylitol by the simultaneous co-utilization of cellodextrin and xylose. Preferably, the cellodextrin is cellobiose.

“Host cell” and “host microorganism” are used interchangeably herein to refer to a living biological cell that can be transformed via insertion of recombinant DNA or RNA. Such recombinant DNA or RNA can be in an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

As used herein, a “corresponding cell” refers to a cell that is substantially similar to a host cell, but that lacks the recombinant polypeptides of the recombinant host cell. Corresponding cells are grown in substantially equivalent growth conditions and environments as host cells. Examples of corresponding cells include wild-type cells of the same species as the recombinant host cells. Such wild-type cells could include the parent of the recombinant host cells. Other corresponding cells include those carrying vector backbones, which have not been substantially modified to encode the recombinant polypeptides described in the present disclosure. Other types of corresponding cells will be apparent to those skilled in the art.

Any prokaryotic or eukaryotic host cell may be used in the present disclosure so long as it remains viable after being transformed with a sequence of nucleic acids. Preferably, the host cell is not adversely affected by the transduction of the necessary nucleic acid sequences, the subsequent expression of the proteins (e.g., xylose reductases, transporters, etc.), or the resulting intermediates. Suitable eukaryotic cells include, without limitation, fungal, plant, insect and mammalian cells.

In preferred embodiments, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In certain embodiments, the fungal cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of the present disclosure, yeast shall be defined as described in Biology and Activities of Yeast (Skinner, F. A., Passmore, S. M., and Davenport, R. R., eds, Soc. App. Bacteriol. Symposium Series No. 9, 1980).

In some embodiments, the yeast host is a Candida, Hansenula, Issatchenkia, Kluyveromyces, Pichia, Saccharomyces, Schizosaccharomyces, or Yarrowia strain.

In other embodiments, the yeast host is a Saccharomyces carlsbergensis (Todkar, 2010), Saccharomyces cerevisiae (Duarte et al., 2009), Saccharomyces diastaticus, Saccharomyces douglasii, Saccharomyces kluyveri, Saccharomyces norbensis, Saccharomyces monacensis (GB-Analysts Reports, 2008), Saccharomyces bayanus (Kristen Publicover, 2010), Saccharomyces pastorianus (Nakao et al., 2007), Saccharomyces pombe (Mousdale, 2008), or Saccharomyces oviformis strain. In yet other embodiments, the yeast host is Kluyveromyces lactis (O. W. Merten, 2001), Kluyveromyces fragilis (Pestal et al., 2006; Siso, 1996), Kluyveromyces marxiamus (K. Kourkoutas et al., 2008), Pichia stipitis (Almeida et al., 2008), Candida shehatae (Ayhan Demirbas, 2003), or Candida tropicalis (Jamai et al., 2006). In other embodiments, the yeast host may be Yarrowia lipolytica (Biryukova E. N., 2009), Brettanomyces custersii (Spindler D. D. et al., 1992), or Zygosaccharomyces roux (Chaabane et al., 2006). In one preferred embodiment, the yeast cell is S. cerevisiae.

In other embodiments, the fungal host is a filamentous fungal strain. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In some embodiments, the filamentous fungal host is, without limitation, an Acremonium, Aspergillus, Fusarium, Humicola, Mucor, Myceliophthora, Neurospora, Penicillium, Scytalidium, Thielavia, Tolypocladium, or Trichoderma strain.

In other embodiments, the filamentous fungal host is an Aspergillus awamori, Aspergillus foetidus, Aspergillus japonicus, Aspergillus nidulans, Aspergillus niger, or Aspergillus oryzae strain. In still other embodiments, the filamentous fungal host is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum strain. In yet other embodiments, the filamentous fungal host is a Humicola insolens, Humicola lanuginosa, Mucor miehei, Myceliophthora thermophila, Neurospora crassa, Penicillium purpurogenum, Scytalidium thermophilum, Sporotrichum thermophile (Topakas et al., 2003), or Thielavia terrestris strain. In a further embodiment, the filamentous fungal host is a Trichoderma harzianum, Trichoderma koningii, Trichoderma longibrachiatum, Trichoderma reesei, or Trichoderma viride strain.

In other embodiments, the host cell is prokaryotic, and in certain embodiments, the prokaryotes are E. coli (Dien, B. S. et al., 2003; Yomano, L. P. et al., 1998; Moniruzzaman et al., 1996), Bacillus subtilis (Susana Romero et al., 2007), Zymomonas mobilis (B. S. Dien et al., 2003; Weuster Botz, 1993; Alterthum and Ingram, 1989), Clostridium sp. (Zeikus, 1980; Lynd et al., 2002; Demain et al., 2005), Clostridium phytofermentans (Leschine S., 2010), Clostridium thermocellum (Lynd et al., 2002), Clostridium beijerinckii (Giles Clark, 2008), Clostridium acetobutylicum (Moorella thermoacetica) (Huang W. C. et al., 2004; Dominik et al., 2007), Thermoanaerobacterium saccharolyticum (Marietta Smith, 2009), or Klebsiella oxytoca (Dien, B. S. et al., 2003; Zhou et al., 2001; Brooks and Ingram, 1995). In other embodiments, the prokaryotic host cells are Carboxydocella sp. (Dominik et al., 2007), Corynebacterium glutamicum (Masayuki Inui, et al., 2004), Enterobacteriaceae (Ingram et al., 1995), Erwinia chrysanthemi (Zhou and Ingram, 2000; Zhou et al., 2001), Lactobacillus sp. (McCaskey, T. A., et al., 1994), Pediococcus acidilactici (Zhou, S. et al., 2003), Rhodopseudomonas capsulata (X. Y. Shi et al., 2004), Streptococcus lactis (J. C. Tang et al., 1988), Vibrio furnissii (L. P. Wackett, 2010), Vibrio furnissii M1 (Park et al., 2001), Caldicellulosiruptor saccharolyticus (Z. Kadar et al., 2004), or Xanthomonas campestris (S. T. Yang et al., 1987). In other embodiments, the host cells are cyanobacteria. Additional examples of bacterial host cells include, without limitation, those species assigned to the Escherichia, Enterobacter, Azotobacter, Erwinia, Bacillus, Pseudomonas, Klebsiella, Proteus, Salmonella, Serratia, Shigella, Rhizobia, Vitreoscilla, Synechococcus, Synechocystis, and Paracoccus taxonomical classes.

The host cells of the present disclosure may be genetically modified in that recombinant nucleic acids have been introduced into the host cells, and as such the genetically modified host cells do not occur in nature. A suitable host cell of the present disclosure is one capable of expressing one or more nucleic acid constructs encoding one or more proteins for different functions.

“Recombinant nucleic acid” or “heterologous nucleic acid” or “recombinant polynucleotide” as used herein refers to a polymer of nucleic acids wherein at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids comprises two or more subsequences that are not found in the same relationship to each other in nature. For example, regarding instance (c), a recombinant nucleic acid sequence will have two or more sequences from unrelated genes arranged to make a new functional nucleic acid. Specifically, the present disclosure describes the introduction of an expression vector into a host cell, where the expression vector contains a nucleic acid sequence coding for a protein that is not normally found in a host cell or contains a nucleic acid coding for a protein that is normally found in a cell but is under the control of different regulatory sequences. With reference to the host cell's genome, then, the nucleic acid sequence that codes for the protein is recombinant. A protein that is referred to as recombinant generally implies that it is encoded by a recombinant nucleic acid sequence in the host cell.

In some embodiments, the genes encoding the desired proteins in the host cell may be heterologous to the host cell or these genes may be endogenous to the host cell but are operatively linked to heterologous promoters and/or control regions which result in the higher expression of the gene(s) in the host cell. In certain embodiments, the host cell does not naturally produce the desired proteins, and contains heterologous nucleic acid constructs capable of expressing one or more genes necessary for producing those molecules.

“Endogenous” as used herein with reference to a nucleic acid molecule or polypeptide and a particular cell or microorganism refers to a nucleic acid sequence or polypeptide that is in the cell and was not introduced into the cell using recombinant engineering techniques; for example, a gene that was present in the cell when the cell was originally isolated from nature.

“Genetically engineered” or “genetically modified” refers to any recombinant DNA or RNA method used to create a prokaryotic or eukaryotic host cell that expresses a protein at elevated levels, at lowered levels, or in a mutated form. In other words, the host cell has been transfected, transformed, or transduced with a recombinant polynucleotide molecule, and thereby been altered so as to cause the cell to alter expression of a desired protein. Methods and vectors for genetically engineering host cells are well known in the art; for example various techniques are illustrated in Current Protocols in Molecular Biology, Ausubel et al., eds. (Wiley & Sons, New York, 1988, and quarterly updates). Genetically engineering techniques include, without limitation, expression vectors, and targeted homologous recombination and gene activation (see, for example, U.S. Pat. No. 5,272,071).

### Enzymes of the Present Disclosure

In certain embodiments, the host cells of the present disclosure contain recombinant enzymes.

As used herein, enzymatic reactions can be classified according to their Enzyme Commission (EC) number. The EC number associated with a given enzyme specifies the classification of the type of enzymatic reaction that a given enzyme is capable of catalyzing. EC numbers do not specify identities of enzymes, but instead specify the identity of the chemical reaction that a given enzyme catalyzes. EC classifications are helpful to those skilled in the art in identifying the molecular function and/or activity of a given protein outside of knowing its unique identifying classification with regard to the organism it came from, such as its NCBI (National Council for Biotechnology) identifier.

Xylose Reductases

The host cells of the present disclosure contain a recombinant xylose reductase. As used herein, “xylose reductase” and “XR” refer to an enzyme that catalyzes the following reaction: xylose+NAD(P)H+H+=xylitol+NAD(P)+ (EC 1.1.1.21). Other names for xylose reductase include “aldehyde reductase,” “aldose reductase,” “polyol dehydrogenase (NADP+),” “ALR2,” “NADPH-aldopentose reductase,” “NADPH-aldose reductase,” and “alditol:NAD(P)+1-oxidoreductase.”

Suitable xylose reductases may be obtained from any microorganism capable of metabolizing xylose. Examples of such microorganisms include, without limitation, Pichia sp., Candida sp., Aspergillus sp., and Neurospora crassa. In some embodiments, the xylose reductase is the Pichia stipitis xylose reductase XYL1. In other embodiments, the xylose reductase may be XYL1 from Pichia stipitis (GenBank: ADQ89193.1). Other xylose reductases may include, without limitation, AAF86345.1, AAO91803.1, AEY80024.1, AAC25601.1, and AAW34373.1. Each sequence associated with the foregoing accession numbers is incorporated herein by reference. In other embodiments, the xylose reductase can include one or more Candida boidinii, Candida gilliermondii, Candida parapsilosis, Candida pelliculosa, Candida tropicalis, Neurospora crassa, and Saccharomyces cerevisiae. Other xylose reductases are well-known to those skilled in the art and may be used in the host cells and methods of the present disclosure.

In other embodiments, the xylose reductase is the Saccharomyces cerevisiae aldose reductase GRE3. In some embodiments, the aldose reductase may be GRE3 from Saccharomyces cerevisiae (GenBank: P38715.1). Other aldose reductases (GRE3) may include, without limitation, EDN62343.1, CCG24371.1, NP_{—}011972.1, and CAY80113.1. Each sequence associated with the foregoing accession numbers is incorporated herein by reference. Other aldose reductases are well-known to those skilled in the art and may be used in the host cells and methods of the present disclosure.

In certain embodiments, the xylose reductase has at least about 20%, or at least about 25%, or at least about 30%, or at least about 40%, or at least about 50%, or at least about 55%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or at least about 100% amino acid identity to XYL1 or GRE.

It has been previously shown that the integration of multiple copies of a xylose reductase can improve xylitol productivity as compared to an episomal vector system (see, Chung et al., 2002, Enzyme and microbial technology, 30, 809-816). Accordingly, in some embodiments, the host cells further contain at least one additional recombinant xylose reductase. Preferably, the at least one additional recombinant xylose reductase is XYL1 or GRE.

Cellodextrin Transporters

The host cells of the present disclosure also contain a recombinant cellodextrin transporter. A cellodextrin transporter is any transmembrane protein that transports a cellodextrin molecule from outside of the cell to the inside of the cell and/or from inside of the cell to outside of the cell.

Cellodextrin transporters have been described in US 2011/0020910, which is herein incorporated by reference in its entirety. NCU00801, NCU00809, NCU8114, XP_{—}001268541.1, and LAC2 have all been discovered to encode polypeptides that transport cellodextrins. In preferred embodiments, the host cell contains a cellodextrin transporter encoded by NCU00801, which is also known as CDT-1 or CBT1. In other preferred embodiments, the host cell contains a cellodextrin transporter encoded by NCU08114, which is also known as CDT-2 or CBT2. In certain embodiments, the cellodextrin transporter has at least about 20%, or at least about 25%, or at least about 30%, or at least about 40%, or at least about 50%, or at least about 55%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or at least about 100% amino acid identity to CDT-1 or CDT-2.

β-Glucosidases

The host cells of the invention further contain a recombinant β-glucosidase. The β-glucosidase may be an intracellular β-glucosidase. As used herein, “β-glucosidase” refers to a β-D-glucoside glucohydrolase (E.C. 3.2.1.21), which catalyzes the hydrolysis of terminal non-reducing β-D-glucose residues with the release of β-D-glucose. A β-glucosidase is any enzyme that catalyzes the hydrolysis of terminal non-reducing residues in β-D-glucosides with release of glucose.

β-glucosidases have been described in US 2011/0020910, which is herein incorporated by reference in its entirety. In preferred embodiments, the β-glucosidase is a glycosyl hydrolase family 1 member. Members of this group can be identified by the motif, [LIVMFSTC]-[LIVFYS]-[LIV]-[LIVMST]-E-N-G-[LIVMFAR]-[CSAGN]. Here, E is the catalytic glutamate (webpage expasy.org/cgi-bin/prosite-search-ac?PDOC00495). In preferred embodiments, the β-glucosidase is from N. crassa, and in particularly preferred embodiments, the β-glucosidase is NCU00130, which is also known as GH1-1. In some embodiments, the β-glucosidase has at least about 20%, or at least about 25%, or at least about 30%, or at least about 40%, or at least about 50%, or at least about 55%, or at least about 60%, or at least about 65%, or at least about 70%, or at least about 75%, or at least about 80%, or at least about 85%, or at least about 90%, or at least about 91%, or at least about 92%, or at least about 93%, or at least about 94%, or at least about 95%, or at least about 96%, or at least about 97%, or at least about 98%, or at least about 99%, or at least about 100% amino acid identity to GH1-1.

In other embodiments, the β-glucosidase may be an ortholog of NCU00130. Examples of orthologs of NCU00130 include, without limitation, T. melanosporum, CAZ82985.1; A. oryzae, BAE57671.1; P. placenta, EED81359.1; P. chrysosporium, BAE87009.1; Kluyveromyces lactis, CAG99696.1; Laccaria bicolor, EDR09330; Clavispora lusitaniae, EEQ37997.1; and Pichia stipitis, ABN67130.1. Other β-glucosidases that may be used include those from the glycosyl hydrolase family 3. These β-glucosidases can be identified by the following motif according to PROSITE: [LIVM](2)-[KR]-x-[EQKRD]-x(4)-G-[LIVMFTC]-[LIVT]-[LIVMF]-[ST]-D-x(2)-[SGADNIT]. Here D is the catalytic aspartate. Typically, any β-glucosidase may be used that contains the conserved domain of β-glucosidase/6-phospho-β-glucosidase/β-galactosidase found in NCBI sequence COG2723. Specific β-glucosidases may be preferred depending on the cellodextrin transporter contained in the host cell.

Cellobiose and Cellodextrin Phosphorylases

In another aspect of the present disclosure, the host cells contain an intracellular cellobiose phosphorylase or cellodextrin phosphorylase instead of an intracellular β-glucosidase.

When the host cell contains an intracellular β-glucosidase, the β-glucosidase hydrolyzes the cellodextrins to form glucose. The resulting glucose must be phosphorylated using ATP as a phosphate donor in order to be used by the cell.

An alternative mechanism for utilizing cellodextrins may instead rely on cellodextrin phosphorylase (EC 2.4.1.49) or cellobiose phosphorylase (EC 2.4.1.20). These enzymes use phosphate to cleave the beta-glucosidic linkage between glucose moieties in cellodextrins. The phosphorolysis reaction saves 1 ATP equivalent per cleavage reaction and results in the release of glucose-1-phosphate. The resulting glucose-1-phosphate can then be converted to glucose-6-phosphate by phosphoglucomutases (EC 5.4.2.2). The glucose-6-phosphate is then used directly by the organism for growth or fermentation.

Additional Polypeptides

In a further aspect of the present disclosure, the host cells may further contain one or more recombinant polypeptides selected from glucose 6-phosphate dehydrogenases, phosphoglucose isomerases, acetyl-CoA synthetases, isocitrate dehydrogenases, and acetaldehyde dehydrogenases.

In certain embodiments of the present disclosure, the host cells further contain a recombinant glucose 6-phosphate dehydrogenase. As used herein, “glucose 6-phosphate dehydrogenase” refers to a polypeptide having E.C. 1.1.1.49 activity, which catalyzes the conversion of glucose-6-phosphate into 6-phosphoglucono-δ-lactone. Glucose 6-phosphate dehydrogenase is also a cytosolic enzyme in the pentose phosphate pathway, which supplies reducing energy to cells by maintaining the level of the cofactor NADPH. Moreover, it has been previously shown that overexpressing the glucose 6-phosphate dehydrogenase ZWF1 can result in very high xylitol productivity with glucose limited fed-batch fermentation (Kwon et al., 2006, Journal of Molecular Catalysis B: Enzymatic, 43, 86-89). Accordingly, in certain embodiments, the host cell further contains a recombinant glucose 6-phosphate dehydrogenase. Preferably, the glucose 6-phosphate dehydrogenase is ZWF1. In some embodiments, the glucose 6-phosphate dehydrogenase is ZWF1 from Scheffersomyces (Pichia) stipitis, CBS 6054 (GenBank Accession ABN68020.2). Other glucose-6-phosphate dehydrogenases may include, without limitation, CAA93357.1, CAA40611.1, EEU07329.1, DAA10318.1, and EIW08212.1. Each sequence associated with the foregoing accession numbers is incorporated herein by reference. Other glucose-6-phosphate dehydrogenases are well-known to those skilled in the art and may be used in the host cells and methods of the present disclosure. In other embodiments, the glucose 6-phosphate dehydrogenase is overexpressed.

In some embodiments of the present disclosure, the host cells may further contain an endogenous phosphoglucose isomerase. As used herein, “phosphoglucose isomerase” refers to a glucose-6-phosphate isomerase having E.C. 5.3.1.9 activity, which catalyzes the conversion of glucose-6-phosphate into fructose-6-phosphate. Additionally, it has also been previously shown that xylitol productivity in a host cell can be further improved by combining the overexpression of ZWF1 with reducing the expression of the endogenous phosphoglucose isomerase PGI1 (Oh et al., 2007). Accordingly, in certain embodiments, the host cell further contains a phosphoglucose isomerase. Preferably, the phosphoglucose isomerase is PGI1. In some embodiments, the phosphoglucose isomerase is endogenously expressed in the host cell. In other embodiments, expression of the phosphoglucose isomerase is reduced. Methods of reducing the expression of genes in a host cell are well known in the art, and include, without limitation, mutagenesis of the gene encoding the phosphoglucose isomerase and RNA interference targeting the gene encoding the phosphoglucose isomerase. In some embodiments, the host cells further contain a recombinant phosphoglucose isomerase. In some embodiments, the recombinant phosphoglucose isomerase is a dominant negative protein that interferes with the function of the endogenous phosphoglucose isomerase. In some embodiments, expression of the recombinant phosphoglucose isomerase results in a reduction in phosphoglucose isomerase enzyme activity in the host cells.

In other embodiments of the present disclosure, the host cells further contain a recombinant acetyl-CoA synthetase. As used herein, “acetyl-CoA synthetase” and “acetate-CoA ligase” are used interchangeably and refer to a polypeptide having E.C. 6.2.1.1, which catalyzes the formation of a chemical bond between acetate and coenzyme A (CoA). Moreover, it has been previously shown that overexpressing the acetyl-CoA synthetase ACS1 in host cell resulted in improved xylitol productivity through energetic benefits. Accordingly, in certain embodiments, the host cell further contains a recombinant acetyl-CoA synthetase. Preferably, the acetyl-CoA synthetase is ACS1. Other acetyl-CoA synthetases may include, without limitation, EDN59709.1, ABC87079.1, CAY77590.1, or AAC16713.1. Each sequence associated with the foregoing accession numbers is incorporated herein by reference. Other acetyl-CoA synthetases are well-known to those skilled in the art and may be used in the host cells and methods of the present disclosure. In other embodiments, the acetyl-CoA synthetase is overexpressed.

In some embodiments of the present disclosure, the host cells further contain a recombinant isocitrate dehydrogenase. As used herein, “isocitrate dehydrogenase” refer to a polypeptide having E.C. 1.1.1.41 activity, which catalyzes the following reaction: isocitrate+NAD(P)⁺=2-oxoglutarate+CO₂+NAD(P)H+H+. Preferably, the isocitrate dehydrogenase is IDP2. In some embodiments, the isocitrate dehydrogenase may be IDP2 from Saccharomyces cerevisiae (GenBank: EDN59398.1). Other isocitrate dehydrogenases may include, without limitation, BAA22846.1, ABN65656.1, NP_{—}013275.1, CCG20522.1, and EEU09251.1. Each sequence associated with the foregoing accession numbers is incorporated herein by reference. Other isocitrate dehydrogenases are well-known to those skilled in the art and may be used in the host cells and methods of the present disclosure. In other embodiments, the isocitrate dehydrogenase is overexpressed.

In further embodiments of the present disclosure, the host cells further contain a recombinant acetaldehyde dehydrogenase. As used herein, “acetaldehyde dehydrogenase” refers to a polypeptide having E.C. 1.2.1.10 activity, which catalyzes the oxidation of acetaldehyde into acetic acid. Additionally, it has been previously shown that overexpressing the acetaldehyde dehydrogenase ALD6 in host cells resulted in improved xylitol productivity through NADH cofactor regeneration. Accordingly, in certain embodiments, the host cell further contains a recombinant acetaldehyde dehydrogenase. Preferably, the acetaldehyde dehydrogenase is ALD6. In some embodiments, the acetaldehyde dehydrogenase is ALD6 from Saccharomyces cerevisiae (GenBank: AAB68304.1). Other acetaldehyde dehydrogenases may include, without limitation, P54115.4 and EDN61079.1. Each sequence associated with the foregoing accession numbers is incorporated herein by reference. Other acetaldehyde dehydrogenases are well-known to those skilled in the art and may be used in the host cells and methods of the present disclosure. In other embodiments, the acetaldehyde dehydrogenase is overexpressed.

### Methods of Producing and Culturing Host Cells of the Present Disclosure

Other aspects of the present disclosure relate to the production of host cells containing a recombinant xylose reductase, a recombinant cellodextrin transporter, and a recombinant intracellular β-glucosidase, where the host cell lacks xylitol dehydrogenase and xylulokinase. Such host cells may be used to produce xylitol by the simultaneous co-utilization of cellodextrin and xylose.

Methods of producing and culturing host cells of the present disclosure may include the introduction or transfer of expression vectors containing recombinant polynucleotides into the host cell. Such methods for transferring expression vectors into host cells are well known to those of ordinary skill in the art. For example, one method for transforming E. coli with an expression vector involves a calcium chloride treatment wherein the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Also, microinjection of the nucleic acid sequences provides the ability to transfect host cells. Other means, such as lipid complexes, liposomes, and dendrimers, may also be employed. Those of ordinary skill in the art can transfect a host cell with a desired sequence using these or other methods.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host, is integrated into the genome and replicated together with the chromosome(s) into which it has been integrated. Furthermore, a single vector or plasmid or two or more vectors or plasmids which together contain the total DNA to be introduced into the genome of the host, or a transposon may be used.

The vectors preferably contain one or more selectable markers which permit easy selection of transformed hosts. A selectable marker is a gene the product of which provides, for example, biocide or viral resistance, resistance to heavy metals, prototrophy to auxotrophs, and the like. Selection of bacterial cells may be based upon antimicrobial resistance that has been conferred by genes such as the amp, gpt, neo, and hyg genes.

Suitable markers for yeast hosts are, for example, ADE2, HIS3, LEU2, LYS2, MET3, TRP1, and URA3. Selectable markers for use in a filamentous fungal host include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in Aspergillus are the amdS and pyrG genes of Aspergillus nidulans or Aspergillus oryzae and the bar gene of Streptomyces hygroscopicus. Preferred for use in Trichoderma are bar and amdS.

The vectors preferably contain an element(s) that permits integration of the vector into the host's genome or autonomous replication of the vector in the cell independent of the genome.

For integration into the host genome, the vector may rely on the gene's sequence or any other element of the vector for integration of the vector into the genome by homologous or nonhomologous recombination. Alternatively, the vector may contain additional nucleotide sequences for directing integration by homologous recombination into the genome of the host. The additional nucleotide sequences enable the vector to be integrated into the host genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host. Furthermore, the integrational elements may be non-encoding or encoding nucleotide sequences. On the other hand, the vector may be integrated into the genome of the host by non-homologous recombination.

For autonomous replication, the vector may further comprise an origin of replication enabling the vector to replicate autonomously in the host in question. The origin of replication may be any plasmid replicator mediating autonomous replication which functions in a cell. The term “origin of replication” or “plasmid replicator” is defined herein as a sequence that enables a plasmid or vector to replicate in vivo. Examples of origins of replication for use in a yeast host are the 2 micron origin of replication, ARS1, ARS4, the combination of ARS1 and CEN3, and the combination of ARS4 and CEN6. Examples of origins of replication useful in a filamentous fungal cell are AMA1 and ANSI (Gems et al., 1991; Cullen et al., 1987; WO 00/24883). Isolation of the AMA1 gene and construction of plasmids or vectors comprising the gene can be accomplished according to the methods disclosed in WO 00/24883.

For other hosts, transformation procedures may be found, for example, in Jeremiah D. Read, et al., Applied and Environmental Microbiology, August 2007, p. 5088-5096, for Kluyveromyces; in Osvaldo Delgado, et al., FEMS Microbiology Letters 132, 1995, 23-26, for Zymomonas; in U.S. Pat. No. 7,501,275 for Pichia stipitis; and in WO 2008/040387 for Clostridium.

More than one copy of a gene may be inserted into the host to increase production of the gene product. An increase in the copy number of the gene can be obtained by integrating at least one additional copy of the gene into the host genome or by including an amplifiable selectable marker gene with the nucleotide sequence where cells containing amplified copies of the selectable marker gene, and thereby additional copies of the gene, can be selected for by cultivating the cells in the presence of the appropriate selectable agent.

The procedures used to ligate the elements described above to construct the recombinant expression vectors of the present disclosure are well known to one skilled in the art (see, e.g., Sambrook et al., 1989, supra).

The host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. Methods of the invention may include culturing the host cell such that recombinant nucleic acids in the cell are expressed. For microbial hosts, this process entails culturing the cells in a suitable medium. Typically cells are grown at 35° C. in appropriate media. Preferred growth media in the present invention include, for example, common commercially prepared media such as Luria Bertani (LB) broth, Sabouraud Dextrose (SD) broth or Yeast medium (YM) broth. Other defined or synthetic growth media may also be used and the appropriate medium for growth of the particular host cell will be known by someone skilled in the art of microbiology or fermentation science. Temperature ranges and other conditions suitable for growth are known in the art (see, e.g., Bailey and 011 is 1986).

According to some aspects of the present disclosure, the culture media contains a carbon source for the host cell. Such a “carbon source” generally refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, etc. These include, for example, various monosaccharides such as glucose, oligosaccharides such as cellodextrins, polysaccharides, a biomass polymer such as cellulose or hemicellulose, xylose, arabinose, disaccharides, such as sucrose, saturated or unsaturated fatty acids, succinate, lactate, acetate, ethanol, etc., or mixtures thereof. The carbon source can additionally be a product of photosynthesis, including, but not limited to glucose.

Lignocellulosic biomass is composed of cellulose, hemicellulose, and lignin. In some embodiments, the carbon source is a biomass polymer such as cellulose or hemicellulose. A “biomass polymer” as described herein is any polymer contained in biological material. The biological material may be living or dead. A biomass polymer includes, for example, cellulose, xylan, xylose, hemicellulose, lignin, mannan, and other materials commonly found in biomass. Non-limiting examples of sources of a biomass polymer include grasses (e.g., switchgrass, Miscanthus), rice hulls, bagasse, cotton, jute, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe).

In addition to an appropriate carbon source, media must contain suitable minerals, salts, cofactors, buffers and other components, known to those skilled in the art, suitable for the growth of the cultures and promotion of the enzymatic pathways necessary for the fermentation of various sugars and the production of hydrocarbons and hydrocarbon derivatives. Reactions may be performed under aerobic or anaerobic conditions where aerobic, anoxic, or anaerobic conditions are preferred based on the requirements of the microorganism. As the host cell grows and/or multiplies, expression of the enzymes, transporters, or other proteins necessary for growth on various sugars or biomass polymers, sugar fermentation, or synthesis of hydrocarbons or hydrocarbon derivatives is affected.

### Methods of Producing Xylitol

The present disclosure provides methods of producing xylitol in a host cell. In one aspect, the present disclosure provides a method of producing xylitol, by: a) providing a host cell lacking xylitol deydrogenase and xylulokinase, where the host cell contains a recombinant xylose reductase, a recombinant cellodextrin transporter, and a recombinant β-glucosidase; and b) culturing the host cell in a medium containing cellodextrin and xylose, whereby the host cell produces xylitol from the xylose. Xylitol production may be measured by any method known to one of skill in the art, including without limitation, high performance liquid chromatography.

Culturing conditions sufficient for the host cell to produce xylitol by simultaneously co-utilizing cellodextrin and xylose are well known in the art and include any suitable culturing conditions disclosed herein. Typically, in order to prepare the cellodextrin and xylose contained in the culture medium for co-utilization by the host cell, lignocellulosic biomass is first pretreated to alter its structure and allow for better enzymatic hydrolysis of cellulose. Pretreatment may include physical or chemical methods, including, for example, ammonia fiber/freeze explosion, the lime method based on calcium or sodium hydroxide, and steam explosion with or without an acid catalyst. Acid treatment will release xylose from the hemicellulose component of the lignocellulosic biomass. Next, preferably, the cellulose component of the pretreated biomass is hydrolyzed by a mixture of cellulases. Examples of commercially available cellulase mixtures include Celluclast 1.5L® (Novozymes), Spezyme CP® (Genencor) (Scott W. Pryor, 2010, Appl Biochem Biotechnol), and Cellulyve 50L (Lyven).

Cellulase mixtures typically contain endoglucanases, exoglucanases, and β-glucosidases. In methods of the present disclosure for producing xylitol, the amount of β-glucosidase activity in the cellulase mixture should be minimized as much as possible to prevent cellodextrins such as cellobiose from being degraded into glucose, and thus inhibiting xylose transport into the cell. For example, the culturing medium may contain a cellulase-containing enzyme mixture from an altered organism, where the mixture has reduced β-glucosidase activity compared to a cellulase-containing mixture from an unaltered organism.

The culturing conditions sufficient for the host cell to produce xylitol may include providing cellodextrin and xylose at certain g/L ratios, including, without limitation, a cellodextrin to xylose g/L ratio of 1:1, 1:1.5, 1:2, 1:2.5, 1:3, 1:3.5, 1:4, 1:4.5, 1:5, or higher. In certain preferred embodiments, the medium contains cellodextrin and xylose in a g/L ratio of 1:1, 1:2, or 1:3. Preferably, the medium contains cellodextrin and xylose in a g/L ratio of 1:2.

Additionally, the culturing conditions sufficient for the host cell to produce xylitol may also include culturing the cells at a pH of at least about 5, at least about 5.5, at least about 6, at least about 6.5, or at least about 7. Preferably, the host cells are cultured at a pH of about 6. It should be noted that the pH values described herein may vary by ±0.2. For example a pH value of about 6 could vary from pH 5.8 to pH 6.2.

In some embodiments, culturing a host cell of the present disclosure with cellodextrin and xylose results in a rate of xylitol production that is at least 40%, at least 45%, at least 50%, at least 55%, at least 60%, at least 65%, at least 70%, at least 75%, at least 80%, at least 85%, at least 90%, at least 95%, at least 100%, at least 150%, at least 200%, at least 250%, at least 300%, at least 350%, at least 400%, at least 450%, or at least 500% greater than the rate of xylitol production in a corresponding cell cultured with glucose and xylose. Typically, the host cell cultured with cellodextrin and xylose and the host cell cultured with glucose and xylose will otherwise be identical in genetic background.

The host cells of the present disclosure contain a recombinant xylose reductase, but lack a xylitol dehydrogenase and a xylulokinase. Thus, the host cells can metabolize xylose to xylitol, but cannot further metabolize xylitol to xylulose and phosphorylated xylulose. Accordingly, in certain embodiments, at most 10%, at most 9%, at most 8%, at most 7%, at most 6%, at most 5%, at most 4%, at most 3%, at most 2%, at most 1%, or less of the xylose is converted to xylulose by the host cell.

In other embodiments of the disclosed methods of producing xylitol, the recombinant xylose reductase, the recombinant cellodextrin transporter, and the recombinant intracellular β-glucosidase are overexpressed. In some embodiments, the recombinant xylose reductase is XYL1 or GRE3. In other embodiments, the recombinant xylose reductase is GRE3. In still other embodiments, the recombinant xylose reductase is YXL1. In yet other embodiments, the recombinant cellodextrin transporter is a cellobiose transporter. Preferably, the cellobiose transporter is CDT-1. In some preferred embodiments, the recombinant intracellular β-glucosidase is GH1-1. In further embodiments, the host cell further contains at least one additional recombinant xylose reductase. Preferably, the at least one additional recombinant xylose reductase is XYL1 or GRE3.

In some embodiments of the disclosed methods of producing xylitol, the host cell further contains one or more recombinant polypeptides selected from a glucose 6-phosphate dehydrogenase, an acetyl-CoA synthetase, an isocitrate dehydrogenase, and an acetaldehyde dehydrogenase. Preferably, the glucose 6-phosphate dehydrogenase is ZWF1, the acetyl-CoA synthetase is ACS1, the isocitrate dehydrogenase is IDP2, and the acetaldehyde dehydrogenase is ALD6 In some embodiments, the recombinant glucose 6-phosphate dehydrogenase is overexpressed. In other embodiments, the recombinant acetyl-CoA synthetase is overexpressed. In other embodiments, the isocitrate dehydrogenase is overexpressed. In further embodiments, the recombinant acetaldehyde dehydrogenase is overexpressed. In other embodiments, the host cell further contains a phosphoglucose isomerase. In some embodiments, the phosphoglucose isomerase is endogenously expressed in the host cell. In certain preferred embodiments, expression of the phosphoglucose isomerase is reduced. In some embodiments, the phosphoglucose isomerase is PGI1. In other embodiments, the host cell further contains a recombinant phosphoglucose isomerase. In some embodiments, the recombinant phosphoglucose isomerase is a dominant negative protein that interferes with the function of the endogenous phosphoglucose isomerase. In some embodiments, expression of the recombinant phosphoglucose isomerase results in a reduction in phosphoglucose isomerase enzyme activity in the host cells.

It is to be understood that, while the present disclosure has been described in conjunction with the preferred specific embodiments thereof, the foregoing description is intended to illustrate and not limit the scope of the present disclosure. Other aspects, advantages, and modifications within the scope of the present disclosure will be apparent to those skilled in the art to which the present disclosure pertains.

The following examples are offered to illustrate provided embodiments and are not intended to limit the scope of the present disclosure.

### EXAMPLES

### Introduction

Xylitol, a five-carbon sugar alcohol, has been used as a sugar substitute in food industry because of its low caloric and anti-carcinogenic characteristics. In addition, xylitol can be transformed into various commodity chemicals. Biological processes for producing xylitol from cellulosic hydrolyzates have been developed through metabolic engineering of Saccharomyces cerevisiae. As S. cerevisiae cannot utilize xylose as a carbon source, expression of a xylose reductase gene from Scheffersomyces (Pichia) stipitis is required to enable production of xylitol from xylose with high yields. However, an insufficient supply of NAD(P)H for xylose reductase activity, and the inhibition of xylose uptake by glucose are major constraints for achieving high xylitol productivity.

Accordingly, the following Examples demonstrate the ability of an engineered S. cerevisiae to convert xylose into xylitol by the simultaneous utilization of cellobiose and xylose. The SsXYL1 gene coding for XR from S. stipitis was integrated into the S. cerevisiae genome, and a cellodextrin transporter (cdt-1) and an intracellular β-glucosidase (ghl-1) from Neurospora crassa were introduced using multi-copy plasmids. The resulting engineered S. cerevisiae (D-10-BT) was able to produce xylitol from the simultaneous utilization of cellobiose and xylose. As cellobiose does not repress xylose uptake, co-utilization of cellobiose as a co-substrate supported sufficient regeneration of NAD(P)H for XR activity. As a result, the engineered D-10-BT strain exhibited higher volumetric xylitol productivity (1.50 g/L-h) when cellobiose and xylose were co-consumed, as compared to serial utilization of glucose and xylose. Furthermore, the overexpression of S. cerevisiae ALD6, IDP2, or S. stipitis ZWF1 coding for cytosolic NADP⁺-dependent dehydrogenases increased the intracellular NADPH availability of the D-10-BT strain, which resulted in a 37-63% improvement in xylitol productivity when cellobiose and xylose were co-consumed.

### Example 1

### Construction of an Engineered Strain Capable of Producing Xylitol Via Simultaneous Consumption of Cellobiose and Xylose

This example demonstrates that an S. cerevisiae strain engineered to overexpress the XYL1, cdt-1, and ghl-1 genes is able to produce xylitol by the simultaneous consumption of cellobiose and xylose.

### Materials and Methods

Strains and Plasmid Constructs

The S. cerevisiae strain D452-2 (MATalpha, leu2, his3, ura3, and can1) was used for engineering of cellobiose metabolism and XYL1 gene integration.

The Escherichia coli strain DH5 (F-recA1 endA1 hsdR17 [rK-mK+] supE44 thi-1 gyrA relA1) (Invitrogen, Gaithersburg, Md.) was used for gene cloning and manipulation.

The pYS10 vector containing the Scheffersomyces (Pichia) stipitis XYL1 gene under the control of the S. cerevisiae TDH3 promoter was linearized and transformed into the D452-2 strain. The resulting transformed S. cerevisiae strain D-10 had the XYL1 gene integrated into its genome (Sakai et al., 1990).

For expression of the Neurospora crassa β-glucosidase (ghl-1) and cellodextrin transporter (cdt-1) (Galazka et al., 2010; Ha et al., 2011), two open reading frames (cdt-1 and gh1-1) were placed between the PGK promoter and CYC1 terminator. Each expression cassette of β-glucosidase and cellodextrin transporter was amplified by PCR. Amplification primers contained a SwaI blunt enzyme site. After SwaI enzyme treatment, the two cassettes were cloned into EcoRV-treated pRS425 vector to generate the pRS425-BT vector. Table 1 describes the various strains and plasmids used and/or generated during this study.

TABLE 1
Strains and Plasmids

| Strain or Plasmid | Description |
|---|---|
| Strains | |
| S. cerevisiae D452-2 | MATalpha, leu2, his3, ura3, and can1^{a} |
| S. cerevisiae D-BT | S. cerevisiae D452-2 (pRS425-BT)^{b} |
| S. cerevisiae D-10 | Isogenic of D452-2 except for leu2::LEU2-SsXYL1 |
| S. cerevisiae D-10-BT | S. cerevisiae D-10 (pRS425-BT) |
| S. cerevisiae D-10-BT-C | S. cerevisiae D-10-BT (pRS42KTEF) |
| S. cerevisiae D-10-BT-ALD6 | S. cerevisiae D-10-BT (pRS42KTEF-ALD6) |
| S. cerevisiae D-10-BT-IDP2 | S. cerevisiae D-10-BT (pRS42KTEF-IDP2) |
| S. cerevisiae D-10-BT-SsZWF1 | S. cerevisiae D-10-BT (pRS42KTEF-SsZWF1) |
| Plasmids | |
| pYS10 | pRS305 TDH3_{P}-SsXYL1-TDH3_{T}^{c} |
| pRS425 | LEU2, 2-μm origin^{d} |
| pRS425-BT | gh1-1 and cdt-1 under the control of PGK1 promoter in pRS425^{b} |
| pRS42K | 2-μm origin, drug resistance marker (G418^{R})^{e} |
| pRS42KTEF | 2-μm origin, drug resistance marker (G418^{R}), TEF_{P} and CYC1_{T} |
| pRS42KTEF-ALD6 | ALD6 under the control of TEF promoter in pRS42K |
| pRS42KTEF-IDP2 | IDP2 under the control of TEF promoter in pRS42K |
| pRS42KTEF-SsZWF1 | SsZWF1 under the control of TEF promoter in pRS42K |

In Table 1, unless otherwise noted, all strains and plasmids were generated in this study. Several strains and plasmids were previously generated: ^{a} Hosaka et al. (1992), ^{b} Ha et al. (2011), ^{c} Jin and Jeffries (2003), ^{d} Christianson et al. (1992), ^{e} Taxis and Knop (2006).

Yeast Transformation

Transformation of expression cassettes for constructing xylose and cellobiose metabolic pathways was performed using the yeast EZ-Transformation kit (BIO 101, Vista, Calif.). XYL1 transformants were selected on YSC medium containing 20 g/L glucose. pRS425-BT transformants were selected on YSC medium containing 20 g/L cellobiose Amino acids and nucleotides were added as necessary.

Medium and Culture Conditions

E. coli was grown in Luria-Bertani medium, with 50 μg/mL of ampicillin added to the medium when required.

Yeast strains were routinely cultivated at 30° C. in YP medium (10 g/L yeast extract, 20 g/L Bacto peptone) with 20 g/L glucose.

To select transformants using an amino acid auxotrophic marker, yeast synthetic complete (YSC) medium was used. The YSC medium was supplemented with 6.7 g/L yeast nitrogen base plus 20 g/L glucose, 20 g/L agar, and CSM-Leu-Trp-Ura (Bio 101, Vista, Calif.), which supplied appropriate nucleotides and amino acids.

Preparation of Crude Extract and Xylose Reductase Activity Assay

Yeast cells were grown to mid-log phase at 30° C. in YPD. Cells were harvested by centrifugation at 3000×g for 5 min. The cell pellet was washed and suspended in Y-PER solution. After incubation at room temperature for 20 min, the cell suspension was centrifuged at 1300 rpm for 10 min to remove cell debris completely.

Xylose reductase (EC 1.1.1.21) activity of the crude cell extract was measured in a reaction mixture with the following composition: 50 mM sodium phosphate buffer, pH 6.5, 1 M xylose, 2 mM NADPH (Verduyn et al., 1985). Oxidation of NADPH in the reaction was monitored by a spectrophotometer at 340 nm (Biomate 5, Thermo, NY). One unit of enzyme activity is defined as the amount of enzyme that catalyzes 1 μmol of substrate per min at 30° C. Protein concentration was determined by the BCA method (Pierce, Rockford, Ill.).

Fermentation Experiments

Yeast cells were grown in YP medium containing 20 g/L of cellobiose. Cells at mid-exponential phase were harvested and inoculated after washing twice by sterilized water. Flask fermentation experiments were performed using 50 mL of YP medium containing sugars in 250 mL flask at 30° C. with an initial OD₆₀₀ of ˜1.0, and under oxygen limited conditions.

Analytical Methods

Cell growth was monitored by optical density (OD) at 600 nm using a UV-visible Spectrophotometer (Biomate 5, Thermo, NY).

Ethanol concentration was determined by high performance liquid chromatography (HPLC, Agilent Technologies 1200 Series) equipped with a refractive index detector using a Rezex ROA-Organic Acid H+ (8%) column (Phenomenex Inc., Torrance, Calif.). The column was eluted with 0.005 N of H₂SO₄ at a flow rate of 0.6 ml/min at 50° C.

### Results

As wild-type S. cerevisiae cannot produce xylitol from xylose, an overexpression cassette (pYS10) containing the XYL1 gene under the control of TDH3 promoter and terminator was integrated into the genome of S. cerevisiae D452-2. The resulting transformed strain (D-10) was confirmed by measuring xylose reductase activity (0.95 U/mg of protein).

An expression cassette (pRS425-BT) expressing both cdt-1 and ghl-1 under control of PGK1 promoter and CYC1 terminator was also constructed. While the D-10 strain is not a leucine auxotroph, it was found that the pRS425-BT transformants could be selected on agar plates containing cellobiose as a sole carbon source.

As shown in Table 2, D452-2 cells transformed with the pRS425-BT vector had efficient growth and cellobiose fermentation.

TABLE 2
Efficient cellobiose fermentation by the engineered S. cerevisiae (D-BT)

| D-BT strain | | | | |
|---|---|---|---|---|
| Time (h) | OD | biomass (g/L) | cellobiose (g/L) | ethanol (g/L) |
| 0 | 2.3637 | 0.6855 | 83.64 | 0 |
| 8 | 4.6563 | 1.3503 | 77.5308 | 2.1248 |
| 13 | 7.4052 | 2.1475 | 68.3441 | 4.261 |
| 23 | 16.1942 | 4.6963 | 16.2031 | 19.2809 |
| 30 | 20.4578 | 5.9328 | 2.8724 | 28.3887 |
| 36 | 22.0286 | 6.3883 | 1.8181 | 32.1761 |
| 47 | 23.6555 | 6.8601 | 1.1766 | 32.8462 |

In order to produce xylitol via simultaneous utilization of cellobiose and xylose, the pRS425-BT was also introduced into the D-10 strain. The D-10-BT strain was not able to assimilate xylose as a carbon source because xylitol dehydrogenase (XDH) for converting xylitol into xylulose was not introduced (Table 3). Therefore, the D-10-BT strain can be used to produce xylitol without assimilation of xylose using cellobiose as a co-substrate.

TABLE 3
Xylose assimilation by S. cerevisiae
D-10-BT strain as a sole carbon source

| D-10-BT | | | | |
|---|---|---|---|---|
| Time (h) | OD | biomass (g/L) | xylose (g/L) | xylitol (g/L) |
| 0 | | 0.2938 | 48.5967 | 0 |
| 13 | 1.6812 | 0.4875 | 47.4219 | 0.9747 |
| 24 | 1.6257 | 0.4715 | 46.9717 | 1.209 |
| 35 | 1.6153 | 0.4684 | 46.8045 | 1.3906 |
| 48 | 1.5858 | 0.4599 | 46.2238 | 1.5407 |

### Example 2

### Comparisons of Xylitol Production Between Sequential Utilization of Glucose and Xylose and Simultaneous Co-Utilization of Cellobiose and Xylose

This example compares the kinetics of xylitol production by sequential utilization of glucose and xylose and by simultaneous co-utilization of cellobiose and xylose.

### Materials and Methods

Fermentation Experiments

Yeast cells were grown in YP medium containing 20 g/L of cellobiose or glucose to prepare inoculums for xylitol production. Cells at mid-exponential phase were harvested and inoculated after washing twice by sterilized water. Fermentation experiments were performed using 50 mL of YP medium containing sugars in 250 mL flask at 30° C. with an initial OD₆₀₀ of ˜1.0, and under oxygen limited conditions.

Analytical Methods

Cell growth was monitored by optical density (OD) at 600 nm using a UV-visible Spectrophotometer (Biomate 5, Thermo, NY).

Glucose, cellobiose, xylose, xylitol, and ethanol concentrations were determined by high performance liquid chromatography (HPLC, Agilent Technologies 1200 Series) equipped with a refractive index detector using a Rezex ROA-Organic Acid H+ (8%) column (Phenomenex Inc., Torrance, Calif.). The column was eluted with 0.005 N of H₂SO₄ at a flow rate of 0.6 ml/min at 50° C.

The xylitol yield and productivity were determined as follows:

Xylitol yield: (xylitol production)/(consumed xylose concentration)

At a certain point in time, the amount of xylitol production divided by the amount of xylose which the strain consumes during fermentation

Xylitol productivity: (xylitol production)/(fermentation time)

At a certain point in time, the amount of xylitol production divided by the process time.

### Results

The xylitol production profiles of two engineered S. cerevisiae strains was investigated by determining the kinetics of xylitol production by both sequential utilization of glucose and xylose and by simultaneous co-utilization of cellobiose and xylose, batch fermentation experiments were performed in YP medium containing 20 g/L of xylose and 20 g/L of either glucose or cellobiose as a co-substrate (Tables 4 and 5). The two engineered S. cerevisiae strains were the D-10 strain, which expresses the P. stipitis XYL1 gene, and the progeny strain D-10-BT, which expresses both cdt-1 and ghl-1 in addition to XYL1.

TABLE 4
Xylose consumption and xylitol production during serial utilization
of glucose and xylose by strain D-10

| D-10 | Biomass | Glucose | | | Ethanol |
|---|---|---|---|---|---|
| Time (h) | (g/L) | (g/L) | Xylose (g/L) | Xylitol (g/L) | (g/L) |
| 0 | 0.4901 | 20.06 | 20.8 | 0 | 0 |
| 12 | 2.6264 | 0 | 16.3398 | 3.2461 | 8.4168 |
| 24 | 2.8604 | 0 | 12.116 | 7.7748 | 7.4843 |
| 36 | 2.9032 | 0 | 9.0092 | 11.5483 | 6.2948 |
| 48 | 2.9157 | 0 | 7.0703 | 13.4237 | 5.4294 |

In Table 4, data values are averages of two independent experiments.

As expected, the D-10 strain consumed glucose first and then converted xylose into xylitol afterwards, as glucose inhibits xylose uptake. In 48 h, the D-10 strain produced 13 g/L of xylitol by utilizing 3 g/L of produced ethanol for cofactor regeneration (Table 4). The xylitol yield of the D10 strain was 0.98 g xylitol/g xylose, while the xylitol productivity was 0.28 g/L-h (Table 4).

In contrast to the D10 strain, the D-10-BT strain was able to co-utilize cellobiose and xylose simultaneously, as cellobiose does not inhibit xylose uptake. After consuming all initially added sugars (20 g/L of cellobiose and 20 g/L of xylose), the D-10-BT produced 19 g/L of xylitol with higher cell mass than the D-10 strain using glucose as a co-substrate within 48 h (Table 5). This resulted in a xylitol yield of 1.00 g/g and a xylitol productivity of 0.40 g/L-h.

TABLE 5
Xylose consumption and xylitol production during simultaneous
co-utilization of cellobiose and xylose by strain D-10-BT

| D-10-BT | Biomass | Cellobiose | | | Ethanol |
|---|---|---|---|---|---|
| Time (h) | (g/L) | (g/L) | Xylose (g/L) | Xylitol (g/L) | (g/L) |
| 0 | 0.4307 | 19.5877 | 20.3466 | 0 | 0 |
| 12 | 1.3484 | 15.4924 | 14.0125 | 5.0022 | 0 |
| 24 | 2.4202 | 6.9377 | 6.3168 | 12.5233 | 2.9932 |
| 36 | 3.3082 | 2.2771 | 2.5479 | 17.1008 | 4.7783 |
| 48 | 3.7158 | 0.3796 | 1.0532 | 19.399 | 4.9686 |

In Table 5, data values are averages of two independent experiments.

These results indicate that the introduced genes (XYL1, cdt-1, and ghl-1) were functionally expressed and that the phenotypes of the engineered strains were consistent with their genotypes. As previously reported, strong glucose repression on xylitol production was observed when a mixture of glucose and xylose was used. In fact, ethanol produced from glucose fermentation was used as a co-substrate for xylitol production (Table 4). However, there was no inhibition by cellobiose on xylitol production when a mixture of cellobiose and xylose was used (Table 5).

### Example 3

### Enhanced Xylitol Production Through Simultaneous Co-Utilization of Cellobiose and Xylose

This example demonstrates that increasing initial xylose and cellobiose concentrations improved xylitol yield and productivity.

### Materials and Methods

Fermentation Experiments

Yeast cells were grown in YP medium containing 20 g/L of cellobiose or glucose to prepare inoculums for xylitol production. Cells at mid-exponential phase were harvested and inoculated after washing twice by sterilized water. Fermentation experiments were performed using 50 mL of YP medium containing sugars in 250 mL flask at 30° C. with an initial OD₆₀₀ of ˜1.0, and under oxygen limited conditions.

Analytical Methods

Cell growth was monitored by optical density (OD) at 600 nm using a UV-visible Spectrophotometer (Biomate 5, Thermo, NY).

Cellobiose, xylose, xylitol, and ethanol concentrations were determined by high performance liquid chromatography (HPLC, Agilent Technologies 1200 Series) equipped with a refractive index detector using a Rezex ROA-Organic Acid H+ (8%) column (Phenomenex Inc., Torrance, Calif.). The column was eluted with 0.005 N of H₂SO₄ at a flow rate of 0.6 ml/min at 50° C. The final pH was determined using the following protocol:

1. Take the sample from the fermentation broth
2. Centrifugation to get supernatant
3. Place pH probe in the supernatant
4. Measure pH using a Beckman Instruments, Inc. Φ390 pH meter

### Results

When 30 g/L of xylose and 34 g/L of cellobiose were added initially, dry cell mass increased up to 4.4 g/L and 30 g/L of xylitol was produced by simultaneous co-utilization of cellobiose and xylose within 62 h (Table 6). The xylitol productivity was 0.49 g xylitol/L-h and the xylitol yield was 1.00 g xylitol/g xylose.

TABLE 6
Xylitol production through simultaneous co-utilization
of 30 g/L xylose and 34 g/L cellobiose

| | D-10-BT | | | | | |
|---|---|---|---|---|---|---|
| | Cellobiose | Xylose | Biomass | Xylitol | Ethanol | Acetate |
| Time (h) | (g/L) | (g/L) | (g/L) | (g/L) | (g/L) | (g/L) |
| 0 | 33.8533 | 29.8409 | 0.3109 | 0 | 0 | 0 |
| 12 | 29.9161 | 23.9082 | 1.4189 | 4.9746 | 0 | 0.2427 |
| 24 | 18.0874 | 12.6186 | 2.6416 | 15.2729 | 2.8365 | 0.4288 |
| 36 | 9.9266 | 5.3143 | 3.512 | 23.5793 | 4.999 | 0.6537 |
| 48 | 6.7198 | 2.0835 | 4.1057 | 27.9417 | 5.4155 | 0.7696 |
| 62.5 | 2.9434 | 0.7624 | 4.3221 | 30.4925 | 5.6149 | 0.6213 |

When the initial concentration of cellobiose was increased to 44 g/L and that of xylose was increased to 40 g/L, 39 g/L of xylitol was produced within 72 h, resulting in higher xylitol productivity (0.55 g xylitol/L-h) with the same xylitol yield (1.00 g xylitol/g xylose) (Table 7).

TABLE 7
Xylitol production through simultaneous co-utilization
of 40 g/L xylose and 44 g/L cellobiose

| | D-10-BT | | | | | |
|---|---|---|---|---|---|---|
| | Cellobiose | Xylose | Biomass | Xylitol | Ethanol | Acetate |
| Time (h) | (g/L) | (g/L) | (g/L) | (g/L) | (g/L) | (g/L) |
| 0 | 44.4183 | 39.652 | 0.3109 | 0 | 0 | 0 |
| 12 | 42.1138 | 34.913 | 1.3031 | 4.7115 | 0 | 0.2987 |
| 24 | 30.8063 | 23.3413 | 2.2491 | 14.2117 | 2.5527 | 0.5086 |
| 36 | 22.8454 | 14.4327 | 2.8176 | 23.7885 | 3.6709 | 0.8623 |
| 48 | 20.5748 | 8.852 | 3.195 | 31.624 | 3.8305 | 1.1708 |
| 62.5 | 20.1263 | 3.8075 | 3.3208 | 37.0227 | 3.3574 | 1.4486 |
| 72 | 19.2418 | 2.6072 | 3.3812 | 39.3868 | 2.9234 | 1.4866 |

By increasing the initial concentrations of xylose and cellobiose were increased from 20 g/L to 40 g/L, 33% higher xylitol productivity (0.40 vs. 0.55 g/L-h) was observed when cellobiose and xylose were co-utilized simultaneously. As shown in Tables 6 and 7, more acetate accumulation was observed when higher sugar concentrations were used. The accumulated acetate might have inhibited the cell growth as proposed previously (Nair and Zhao et al., 2010) With higher initial sugar concentrations (44 g/L of cellobiose and 40 g/L of xylose), cell growth was less than those with lower sugar concentrations (13 and 15), because cellobiose consumption was stopped at the middle of fermentation. At that point, the pH was decreased to 4.5, while using lower sugar concentrations resulted in a final pH that was above 5.0. In addition, through 25 g/L of cellobiose consumption, 39 g/L of xylose was efficiently converted to xylitol. These results suggest that maintaining the culture medium at a pH above 5.0 results in efficient cellobiose fermentation. Moreover, smaller amounts of cellobiose would be enough to support cofactor regeneration for maintaining xylose reductase activity.

### Example 4

### Increased Xylitol Productivity in pH Controlled Bioreactor Fermentation

This example demonstrates optimization of xylitol production via simultaneous co-utilization of cellobiose and xylose by performing bioreactor fermentations with different cellobiose and xylose concentration ratios and a constant pH of 6.0.

### Materials and Methods

Fermentation Experiments

Yeast cells were grown in YP medium containing 20 g/L of cellobiose or glucose to prepare inoculums for xylitol production. Cells at mid-exponential phase were harvested and inoculated after washing twice by sterilized water. Fed-batch fermentation was performed using Sixfors Bioreactors (Appropriate Technical Resources, Inc) with 400 ml of working volume at 30° C. and pH 6.0. Agitation speed and aeration rate were 500 rpm and 2 vvm, respectively.

Analytical Methods

Cell growth was monitored by optical density (OD) at 600 nm using a UV-visible Spectrophotometer (Biomate 5, Thermo, NY).

Cellobiose, xylose, xylitol, and ethanol concentrations were determined by high performance liquid chromatography (HPLC, Agilent Technologies 1200 Series) equipped with a refractive index detector using a Rezex ROA-Organic Acid H+ (8%) column (Phenomenex Inc., Torrance, Calif.). The column was eluted with 0.005 N of H₂SO₄ at a flow rate of 0.6 ml/min at 50° C.

### Results

Initially, 22 g/L of cellobiose and 63 g/L of xylose were added to the bioreactor with S. cerevisiae D-10-BT at an initial OD ˜1.0 (Table 8). After consumption of 22 g/L of cellobiose, cell growth increased up to an OD of 19, and 42 g/L of xylose was converted to xylitol within 34 h, resulting in high xylitol productivity (1.26 g/L-h) with an almost theoretical yield (1.00 g/g). Although xylitol productivity increased significantly as compared to flask fermentations, due to pH control, all of the added xylose was not converted into xylitol. This result suggests that the amount of cellobiose (22 g/L) was not enough to support the complete conversion of the added xylose (63 g/L).

TABLE 8
Xylitol production in a bioreactor under pH control using a 1:3 ratio of
cellobiose:xylose

| | Cellobiose | Xylose | | | Ethanol |
|---|---|---|---|---|---|
| Time (h) | (g/L) | (g/L) | Biomass (g/L) | Xylitol (g/L) | (g/L) |
| 0 | 22.3717 | 62.7291 | 0.2494 | 0 | 0 |
| 1.5 | 21.7804 | 62.2922 | 0.2842 | 0.101 | 0 |
| 4 | 19.7233 | 61 | 0.3625 | 0.4763 | 0.9713 |
| 8 | 12.6906 | 58.9479 | 0.8845 | 2.0926 | 1.7265 |
| 12 | 5.781 | 52.5583 | 1.6095 | 7.2162 | 2.0726 |
| 16 | 2.8234 | 44.8698 | 2.436 | 14.2456 | 2.1371 |
| 20 | 0 | 37.4698 | 3.2045 | 22.1768 | 1.6465 |
| 24 | 0 | 31.155 | 3.9875 | 29.5206 | 1.1768 |
| 28 | 0 | 25.4739 | 4.64 | 36.543 | 0 |
| 34 | 0 | 20.4698 | 5.3795 | 42.7408 | 0 |

As the ratio of 1:3 between cellobiose and xylose was too low to support complete xylitol production, a mixture of cellobiose and xylose with a ratio of 1:2 was used. Moreover, the xylose concentration was increased to 102 g/L. Therefore, 46 g/L of cellobiose and 102 g/L of xylose were initially added to the bioreactor with the D-10-BT strain.

During the fermentation, depletion of cellobiose at 24 h was observed; however, not all xylose was converted into xylose. Accordingly, 16 g/L of cellobiose was added at 28 h to complete the conversion of xylose into xylitol (Table 9). The cell mass increased up to 8.6 g/Land 97 g/L of consumed xylose was converted to 93 g/L of xylitol after consumption of 62 g/L of cellobiose. As a result, a high xylitol productivity (1.50 g/L-h) was achieved even though lower initial inoculums were used without utilizing the sophisticated feeding strategies that are generally required when using mixtures of glucose and xylose.

TABLE 9
Xylitol production in a bioreactor under pH control using a 1:2 ratio of
cellobiose:xylose

| | Cellobiose | Xylose | | | Ethanol |
|---|---|---|---|---|---|
| Time (h) | (g/L) | (g/L) | Biomass (g/L) | Xylitol (g/L) | (g/L) |
| 0 | 46.294 | 99 | 1.4906 | 0 | 0 |
| 5 | 30.6185 | 96.05 | 1.6675 | 2.8369 | 0.6649 |
| 10 | 14.3711 | 92.5657 | 2.3925 | 6.9784 | 2.7819 |
| 14 | 6.4278 | 84.3056 | 3.2915 | 13.8216 | 5.2065 |
| 18 | 0 | 75.4131 | 4.031 | 22.8546 | 6.2069 |
| 24 | 0 | 63.9563 | 4.6835 | 35.8512 | 5.3249 |
| 29 | 0 | 54.3918 | 5.0605 | 45.4013 | 4.5506 |
| 29.1 | 15.59 | 52.7019 | 5.017 | 45.7322 | 4.6365 |
| 33.5 | 2.1918 | 39.009 | 5.887 | 56.8799 | 9.5902 |
| 38 | 0 | 32.1494 | 6.38 | 62.5452 | 9.1946 |
| 42 | 0 | 25.064 | 6.641 | 68.8 | 9.3818 |
| 48.5 | 0 | 15.8962 | 7.3225 | 79.6973 | 8.2637 |
| 53 | 0 | 10.1381 | 7.395 | 84.8764 | 8.1131 |
| 62 | 0 | 1.6155 | 8.584 | 92.7672 | 5.398 |

An additional fermentation experiment was performed to produce xylitol via sequential utilization of glucose and xylose under the same conditions. Initial sugar concentrations of 45 g/L glucose and 100 g/L of xylose were added with the D-10 strain into a bioreactor. When glucose was depleted, 18 g/L of glucose was added (Table 10). The final cell mass was 5.6 g/L, but only 57 g/L of xylitol was produced from 63 g/L of consumed xylose after consuming 62 g/L of glucose in 62 h. The fermentation by sequential utilization of glucose and xylose resulted in a lower xylitol productivity of 0.92 g/L·h as compared to the productivity (1.50 g/L·h) by co-utilization of cellobiose and xylose.

TABLE 10
Xylitol production from a mixture of glucose and xylose

| | Glucose | Xylose | | | Ethanol |
|---|---|---|---|---|---|
| Time (h) | (g/L) | (g/L) | Biomass (g/L) | Xylitol (g/L) | (g/L) |
| 0 | 44.6536 | 100 | 1.421 | 0 | 0 |
| 5 | 39.3472 | 100 | 1.711 | 0.73 | 1.7237 |
| 10 | 24.8962 | 100 | 2.6245 | 2.9012 | 7.4045 |
| 14 | 4.8121 | 100 | 3.9005 | 8.4001 | 14.2545 |
| 18 | 0 | 91.9319 | 4.263 | 15.241 | 14.374 |
| 24 | 0 | 84.4492 | 4.437 | 21.5345 | 12.5834 |
| 29 | 0 | 79.1769 | 4.4805 | 27.2571 | 10.6021 |
| 29.1 | 18.9678 | 77.6317 | 4.5385 | 26.6899 | 10.5222 |
| 33.5 | 0 | 69.0573 | 5.0315 | 33.8227 | 17.3681 |
| 38 | 0 | 64.2052 | 5.22 | 36.5577 | 16.3553 |
| 42 | 0 | 59.7649 | 5.2925 | 40.3786 | 15.092 |
| 48.5 | 0 | 52.1157 | 5.4375 | 45.3698 | 15.092 |
| 53 | 0 | 46.9523 | 5.4665 | 48.8166 | 12.3408 |
| 62 | 0 | 37 | 5.626 | 57.3383 | 7.0352 |

### Example 5

### Overexpression of NADPH Regenerating Enzymes and Impact on Xylitol Production

This example demonstrates that overexpression of NADP⁺-dependent dehydrogenases increases the intracellular NADPH availability and results in improved xylitol productivity when cellobiose and xylose are co-consumed.

### Materials and Methods

Strains and Plasmid Construction

To overexpress NADP⁺-dependent dehydrogenases, the ALD6 (S. cerevisiae aldehyde dehydrogenase), IDP2 (S. cerevisiae isocitrate dehydrogenase), and SsZWF1 (S. stipitis glucose-6-phosphate dehydrogenase) genes were cloned separately into the pRS42KTEF plasmid containing the KanMX marker, TEF promoter, and CYC1 terminator. The resulting plasmids (pRS42KTEF-ALD6, pRS42KTEF-IDP2, and pRS42KTEF-SsZWF1) were individually transformed into the S. cerevisiae D-10-BT strain, and the transformants (D-10-BT-C, D-10-BT-ALD6, D-10-BT-IDP2, and D-10-BT-SsZWF1, respectively) were selected on a YP agar plate (10 g/L yeast extract, 20 g/L Bacto peptone, and 20 g/L agar) containing 20 g/L of cellobiose and 200 μg/mL of G418.

Determination of Intracellular Cofactor Concentrations

The NADP⁺ and NADPH concentrations were determined using the NADP⁺/NADPH assay kit which is based on a glucose dehydrogenase cycling reaction (Bioassay Systems, Hayward, Calif.). Exponentially growing cells in YP medium with glucose or cellobiose were harvested, washed with double-distilled water, and used to measure NADP⁺/NADPH concentration according to the manufacturer's protocol.

Fermentation Experiments

Yeast cells were grown in YP medium containing 20 g/L of cellobiose or glucose to prepare inoculums for xylitol production. Cells at mid-exponential phase were harvested and inoculated after washing twice by sterilized water. All of the flask fermentation experiments were performed using 50 mL of YP medium containing sugars in 250 mL flask at 30° C. with initial optical density (OD) at 600 nm of ˜1.0 and under oxygen limited conditions. Fed-batch fermentation was performed using Sixfors Bioreactors (Appropriate Technical Resources, Inc) with 400 ml of working volume at 30° C. and pH 6.0. Agitation speed and aeration rate were 500 rpm and 2 vvm, respectively.

Analytical Methods

Cell growth was monitored by optical density (OD) at 600 nm using a UV-visible Spectrophotometer (Biomate 5, Thermo, NY).

Cellobiose, xylose, xylitol, and ethanol concentrations were determined by high performance liquid chromatography (HPLC, Agilent Technologies 1200 Series) equipped with a refractive index detector using a Rezex ROA-Organic Acid H+ (8%) column (Phenomenex Inc., Torrance, Calif.). The column was eluted with 0.005 N of H₂SO₄ at a flow rate of 0.6 ml/min at 50° C.

### Results

Although the xylose uptake of the co-fermenting strain (D-10-BT) strain was not limited, the intracellular conversion of xylose to xylitol by XR might have been restricted by cytosolic NAD(P)H concentrations. In order to examine if the cofactor availability limits the rate of xylitol production of the co-fermenting strain (D-10-BT), NADP⁺-dependent dehydrogenases were overexpressed in the D-10-BT strain, and monitored the changes in the intracellular NADPH concentrations and xylitol production profiles. The ALD6, IDP2, and SsZWF1 genes, coding for the major NADP⁺-dependent dehydrogenases in S. cerevisiae or S. stipitis (Minard and McAlister-Henn, 2005; Miyagi et al., 2008), were selected as the overexpression targets.

When fermenting 30 g/L of glucose or 30 g/L of cellobiose as a sole carbon source, the D-10-BT strains overexpressing ALD6, IDP2, and SsZWF1 (the D-10-BT-ALD6, D-10-BT-IDP2, and D-10-BT-SsZWF1 strains, respectively) showed approximately 10% higher intracellular NADPH concentrations than the control strain (D-10-BT-C) (Table 11). While the overexpression of ALD6, IDP2, and SsZWF1 increased the intracellular NADPH availability regardless of sugar conditions, the xylitol production profiles did not change when glucose was used as a co-substrate (Table 12). However, the overexpression of the NADP⁺-dependent dehydrogenase genes altered the xylitol production profiles when a mixture of cellobiose and xylose (30 g/L of cellobiose and 30 g/L of xylose) was used (Table 13).

TABLE 11
Ratio of NADPH to NADP⁺ cofactors in engineered S. cerevisiae
D-10-BT strains with NADPH regeneration using a sole carbon source

NADPH:NADP^{+a}

| Strains | Glucose | Cellobiose |
|---|---|---|
| D-10-BT-C | 1.03 ± 0.01 | 1.04 ± 0.01 |
| D-10-BT-ALD6 | 1.12 ± 0.04 | 1.16 ± 0.03 |
| D-10-BT-IDP2 | 1.15 ± 0.00 | 1.13 ± 0.01 |
| D-10-BT-SsZWF1 | 1.09 ± 0.02 | 1.13 ± 0.00 |

In Table 11, “^{a}” means that cells were harvested at mid-exponential phase before depletion of the sole carbon source (glucose or cellobiose) in YP medium to measure the ratios of NADPH to NADP+ cofactors. Values are shown with standard deviation.

TABLE 12
Effects of overexpression of NADPH regenerating enzymes on
xylitol production during growth on glucose and xylose

| | Xylitol Production (g/L) | | | |
|---|---|---|---|---|
| Time | | | | D-10-BT-Ss |
| (h) | D-10-BT-C | D-10-BT-ALD6 | D-10-BT-IDP2 | ZWF1 |
| 0 | 0 | 0 | 0 | 0 |
| 12 | 4.2475 | 4.6965 | 4.106 | 3.7903 |
| 24 | 9.1926 | 10.5166 | 9.4155 | 10.3398 |
| 36 | 13.611 | 12.9486 | 14.0169 | 14.5857 |
| 48 | 15.6914 | 14.146 | 16.1909 | 16.5297 |
| 60 | 17.3305 | 14.994 | 17.6582 | 18.3367 |

Table 12 contains xylitol production data in the various yeast strains from fermentation of a sugar mixture containing 30 g/L of glucose and 30 g/L of xylose.

TABLE 13
Effects of overexpression of NADPH regenerating enzymes on
xylitol production during growth on cellobiose and xylose

| | Xylitol Production (g/L) | | | |
|---|---|---|---|---|
| Time | | | | D-10-BT-Ss |
| (h) | D-10-BT-C | D-10-BT-ALD6 | D-10-BT-IDP2 | ZWF1 |
| 0 | 0 | 0 | 0 | 0 |
| 13 | 2.324 | 3.6483 | 4.969 | 2.9803 |
| 25 | 4.9343 | 8.7273 | 18.1431 | 7.8967 |
| 38 | 10.9973 | 19.4339 | 28.25 | 16.8908 |
| 48 | 18.2488 | 25.4826 | 29.7233 | 24.9453 |
| 62 | 27.3955 | 28.7744 | 30.4136 | 29.6138 |

Table 13 contains xylitol production data in the various yeast strains from fermentation of a sugar mixture containing 30 g/L of cellobiose and 30 g/L of xylose.

When cellobiose was used as a co-substrate, the overexpression of ALD6, IDP2, and SsZWF1 in the D-10-BT strain resulted in a 37-63% improvement in xylitol productivity (Table 14). The best results (0.62 g xylitol/L·h) from the co-fermentation of cellobiose and xylose were achieved by overexpressing the IDP2 gene, which also increased the final biomass by 200%. These results suggest that the intracellular NADPH concentration might limit the xylitol production during the co-fermentation of cellobiose whereas inefficient xylose uptake during the sequential utilization of glucose and xylose might limit xylitol production more dominantly than the NADPH availability.

TABLE 14
Fermentation parameters^{a} of S. cerevisiae D-10-BT strains with NADPH
regeneration

| | Glucose and Xylose | | | Cellobiose and Xylose | | |
|---|---|---|---|---|---|---|
| Strains | X_{Final} | P_{XtOH} | Y_{XtOH} | X_{Final} | P_{XtOH} | Y_{XtOH} |
| D-10-BT-C | 3.56 ± 0.03 | 0.33 ± 0.01 | 0.95 ± 0.01 | 4.14 ± 0.06 | 0.38 ± 0.00 | 1.00 ± 0.00 |
| D-10-BT-ALD6 | 2.92 ± 0.06 | 0.29 ± 0.00 | 0.96 ± 0.01 | 3.62 ± 0.21 | 0.53 ± 0.01 | 1.00 ± 0.00 |
| D-10-BT-IDP2 | 3.64 ± 0.09 | 0.34 ± 0.01 | 0.95 ± 0.02 | 8.33 ± 0.36 | 0.62 ± 0.01 | 1.00 ± 0.00 |
| D-10-BT-SsZWF1 | 3.58 ± 0.04 | 0.34 ± 0.01 | 0.96 ± 0.04 | 5.30 ± 0.00 | 0.52 ± 0.00 | 1.00 ± 0.00 |

Table 14 demonstrates various parameters assayed in the recombinant yeast strains grown on either glucose/xylose or cellobiose/xylose: ^{a} Fermentation parameters: X_{Final}, final cell mass (g cells/L); P_{XtOH}, xylitol productivity (g xylitol/L·h); Y_{XtOH}, xylitol yield (g xylitol/g xylose). Values are shown with standard deviation.

### Discussion

There are at least two major limitations in xylitol production from xylose: intracellular xylose concentration and availability of NADPH. Engineered S. cerevisiae strains used for xylitol production generally utilize glucose as a co-substrate to support cofactor regeneration to maintain xylose reductase activity. As xylose uptake by an engineered S. cerevisiae strain is severely inhibited by glucose, glucose limited fed-batch fermentation is generally performed (Lee et al., 2000). However, glucose limited fed-batch fermentation is very complex and difficult to perform even at small laboratory scales. While glucose limited fed-batch fermentation results in high xylitol yields and high xylitol production productivities, it may not be preferable with large-scale commercial fermentation.

In the above Examples, xylitol production by simple batch fermentations using a mixture of xylose and glucose was compared with a mixture of xylose and cellobiose. Glucose and cellobiose are used as co-substrates that support NADPH regeneration and cell growth. When mixtures of xylose and glucose were used sequential utilization of xylose after glucose depletion was observed, as glucose inhibits xylose uptake. However, when mixtures of xylose and cellobiose were used, simultaneous utilization of xylose and cellobiose was observed because cellobiose does not inhibit xylose uptake.

Simultaneous co-utilization of cellobiose and xylose resulted in 44% higher xylitol productivity than that from serial utilization of glucose and xylose. This higher productivity is thought to be due to two major advantages of simultaneous co-utilization of cellobiose and xylose over sequential utilization of glucose and xylose. First, simultaneous co-utilization of cellobiose and xylose provides a sufficient amount of cofactor (NADPH) for xylose reductase activity, as glucose metabolism may be more efficient than ethanol metabolism at producing NADPH. It is known that the major source of NADPH production in yeast is the glucose-6-phosphate dehydrogenase reaction (Bruinenberg, P. M., de Bot, P. H., van Dijken, J. P., W., Scheffers, A., 1983. The Role of Redox Balances in the Anaerobic Fermentation of Xylose by Yeasts. Eur J Appl Microbiol Biotechnol, 18, 287-292). Second, enhanced cell growth by simultaneous co-utilization increases volumetric xylitol productivity.

When the initial concentrations of xylose and cellobiose were increased from 20 g/L to 30 or 40 g/L, the final concentration of xylitol also increased from 19 g/L to 30 or 39 g/L, resulting in an improvement in xylitol productivity from 0.40 g/L-h to 0.49 or 0.55 g/L-h. Unexpectedly, with the highest initial sugar concentrations (44 g/L of cellobiose and 40 g/L of xylose), cellobiose consumption by the D10-BT strain was stopped in the middle of fermentation (Table 7). It was found that the pH of the culture medium was very low (pH 4.5), suggesting that cellobiose utilization is inhibited at lower pHs. In addition, although a smaller amount of cellobiose (25 g/L) was consumed, all added xylose (39 g/L) was efficiently converted to xylitol suggesting that the ratio of cellobiose and xylose may be changed from 1:1 to 1:2 or 1:3 to support the cofactor regeneration for xylitol production.

In order to maintain culture pH, bioreactor fermentations were performed with pH control at 6.0, and with different ratios of cellobiose and xylose concentrations. When a 1:3 ratio of cellobiose and xylose concentration was used, a total 42 g/L of xylose was converted to xylitol within 34 h. Additionally, xylitol productivity was 1.26 g/L-h with 1.00 g/g of xylitol yield. However, it was found that xylose consumption rates drastically decreased from ˜1.57-1.92 to 0.83 g/L-h at the end of fermentation, while the cell density continued to increase because of ethanol assimilation. This result indicates that cofactor regeneration at the end of fermentation may not be sufficient for efficient xylitol production, as ethanol was being used as a co-substrate after cellobiose depletion.

As such, a 1:2 ratio of cellobiose and xylose was used for the next bioreactor fermentation. Initial concentrations of cellobiose and xylose were increased to 46 g/L and 102 g/L, respectively, and 16 g/L of cellobiose was additionally added when cellobiose was depleted in the middle of fermentation in order to support the cofactor regeneration. 97 g/L of consumed xylose was converted to 93 g/L of xylitol and xylitol productivity increased to 1.50 g/L-h with a xylitol yield of 0.95 g/g. It was found that xylose consumption rates can be maintained at higher levels, 1.27˜2.22 g/L-h, throughout the fermentation. Moreover, the maximum xylose consumption rate (3.11 g/L-h) was observed just after the addition of the 16 g/L of cellobiose in the middle of fermentation. As a result, simultaneous co-utilization of cellobiose and xylose exhibited much higher xylitol productivities than using mixtures of glucose and xylose.

In addition to glucose-limited fed-batch fermentation, numerous metabolic engineering approaches for improving xylitol productivity have been reported. The integration of XYL1 with multiple copies resulted in 1.7 fold improvement in xylitol productivity (1.1 g/L·h) as compared to an episomal vector system (Chung et al., 2002). The overexpression of ZWF1 coding for glucose 6-phosphate dehydrogenase, which could support the NADPH regeneration, resulted in a very high xylitol productivity (2.0 g/L·h) when glucose-limited fed-batch fermentation was performed (Kwon et al., 2006). With the overexpression of ZWF1, the down-regulated expression of PGI1 coding for phosphoglucose isomerase, which catalyzes reversible isomerization of glucose 6-phosphate and fructose 6-phosphate, improved the specific xylitol productivity from 0.28 to 0.34 g/g cells·h (Oh et al., 2007). The overexpression of ACS1, coding for acetyl-CoA synthetase or ALD6, coding for acetaldehyde dehydrogenase improved xylitol productivity 25% or 13%, respectively through energetic benefits or efficient cofactor regeneration (Oh et al., 2012).

The overexpression of ZWF1 or ALD6 (or another NADP⁺-dependent dehydrogenase, IDP2) did not alter the xylitol production profile during the sequential utilization of glucose and xylose, although their overexpression increased the intracellular NADPH concentrations. In contrast, the overexpression of NADP⁺-dependent dehydrogenases resulted in improved xylitol production when cellobiose and xylose were co-consumed (Tables 13 and 14). These results indicated that the xylitol production using glucose as a co-substrate might be limited at the level of xylose uptake, suggesting that glucose-limited fed batch fermentation is necessary to observe beneficial effects of improved NADPH production. However, enhanced NADPH regeneration can lead to improved xylitol production even in batch fermentation of cellobiose and xylose because xylose transport is not inhibited by cellobiose.

The above Examples demonstrate that enhanced xylitol production can be easily achieved through simultaneous co-utilization of cellobiose and xylose even with simple batch or fed-batch fermentations by engineered S. cerevisiae. Through overexpression of several NADPH regenerating genes, xylitol productivity was improved when cellobiose was used as a co-substrate instead of glucose. Moreover, xylitol productivity and/or production via co-fermentation of cellobiose and xylose may be further improved by further overexpressing ZWF1, ACS1, IDP2, and/or ALD6 and reducing the expression of PGI1.

### REFERENCES

1 Christianson, T. W., Sikorski, R. S., Dante, M., Shero, J. H., Hieter, P., 1992. Multifunctional yeast high-copy number shuttle vectors. Gene 110, 119-122.
2 Chung, Y. S., Kim, M. D., Lee, W. J., Ryu, Y. W., Kim, J. H., Seo, J. H., 2002. Stable expression of xylose reductase gene enhances xylitol production in recombinant Saccharomyces cerevisiae. Enzyme and microbial technology, 30, 809-816.
3 Galazka, J. M., Tian, C., Beeson, W. T., Martinez, B., Glass, N. L., Cate, J. H. D., 2010. Cellodextrin transport in yeast for improved biofuel production. Science, 330, 84.
4 Granstrom, T. B., Izumori, K., Leisola, M., 2007. A rare sugar xylitol. Part I: the biochemistry and biosynthesis of xylitol. Applied microbiology and biotechnology, 74, 277-281.
5 Granstrom, T. B., Izumori, K., Leisola, M., 2007. A rare sugar xylitol. Part II:

biotechnological production and future applications of xylitol. Applied microbiology and biotechnology, 74, 273-276.
6 Ha, S. J., Galazka, J. M., Rin Kim, S., Choi, J. H., Yang, X., Seo, J. H., Louise Glass, N., Cate, J. H. D., Jin, Y. S., 2011. Engineered Saccharomyces cerevisiae capable of simultaneous cellobiose and xylose fermentation. Proceedings of the National Academy of Sciences, 108, 504.
7 Hallborn, J., Meinander, N., Hahn-Hegerdal, B., Gorwa, M. F., Pentilla, M., Keranen, S., 1994. The influence of cosubstrate and aeration on xylitol formation by recombinant Saccharomyces cerevisiae expressing the XYL1 gene. Applied microbiology and biotechnology, 42, 326-333.
8 Hallborn, J., Walfridsson, M., Airaksinen, U., Ojamo, H., Hahn-Hagerdal, B., Penttila, M., Keranen, S., 1991. Xylitol production by recombinant Saccharomyces cerevisiae. Nature Biotechnology, 9, 1090-1095.
9 Hosaka, K., Nikawa, J., Kodaki, T., Yamashita, S., 1992. A dominant mutation that alters the regulation of INO1 expression in Saccharomyces cerevisiae. J. Biochem., 111, 352-358.
10 Hyvonen, L., Koivistoinen, P., Voirol, F., 1982. Food technological evaluation of xylitol. Advances in food research, 28, 373-403.
11 Jin, Y.-S., Jeffries, T., 2003. Changing flux of xylose metabolites by altering expression of xylose reductase and xylitol dehydrogenase in recombinant Saccharomyces cerevisiae. Appl. Biochem. Biotechnol. 106, 277-285.
12 Kwon, D. H., Kim, M. D., Lee, T. H., Oh, Y. J., Ryu, Y. W., Seo, J. H., 2006. Elevation of glucose 6-phosphate dehydrogenase activity increases xylitol production in recombinant Saccharomyces cerevisiae. Journal of Molecular Catalysis B: Enzymatic, 43, 86-89.
13 Lee, W. J., Ryu, Y. W., Seo, J. H., 2000. Characterization of two-substrate fermentation processes for xylitol production using recombinant Saccharomyces cerevisiae containing xylose reductase gene. Process Biochemistry, 35, 1199-1203.
14 Makinen, K., 1992. Dietary prevention of dental caries by xylitol-clinical effectiveness and safety. J Appl Nutr, 44, 16-28.
15 Meinander, N. Q., Hahn-Hagerdal, B., 1997. Fed-batch xylitol production with two recombinant Saccharomyces cerevisiae strains expressing XYL1 at different levels, using glucose as a cosubstrate: a comparison of production parameters and strain stability. Biotechnol Bioeng, 54, 391-9.
16 Melaja, A. J., Hamalainen, L., 1977. Process for making xylitol. U.S. Pat. No. 4,008,285.
17 Nair, N. U., Zhao, H., 2010. Selective reduction of xylose to xylitol from a mixture of hemicellulosic sugars. Metab. Eng. 12, 462-468.
18 Minard, K. I., McAlister-Henn, L., 2005. Sources of NADPH in yeast vary with carbon source. J. Biol. Chem. 280, 39890-39896.
19 Miyagi, H., Kawai, S., Murata, K., 2008. Two sources of mitochondrial NADPH in the yeast Saccharomyces cerevisiae. J. Biol. Chem. 284, 7553-7560.
20 Nigam, P., Singh, D., 1995. Processes of fermentative production of Xylitol—a sugar substitute. Process Biochemistry, 30, 117-124.
21 Oh, E.-J., Bae, Y.-H., Kim, K.-H., Park, Y.-C., Seo, J.-H., 2012. Effects of overexpression of acetaldehyde dehydrogenase 6 and acetyl-CoA synthetase 1 on xylitol production in recombinant Saccharomyces cerevisiae. Biocatal. Agric. Biotechnol. 1, 15-19.
22 Oh, Y.-J., Lee, T.-H., Lee, S.-H., Oh, E.-J., Ryu, Y.-W., Kim, M.-D., Seo, J.-H., 2007. Dual modulation of glucose 6-phosphate metabolism to increase NADPH-dependent xylitol production in recombinant Saccharomyces cerevisiae. J. Mol. Catal. B: Enzym. 47, 37-42.
23 Romanos, M. A., Scorer, C. A., Clare, J. J., 1992. Foreign gene expression in yeast: a review. Yeast, 8, 423-488.
24 Sakai, A., Shimizu, Y., Hishinuma, F., 1990. Integration of heterologous genes into the chromosome of Saccharomyces cerevisiae using a delta sequence of yeast retrotransposon Ty. Applied microbiology and biotechnology, 33, 302-306.
25 Taxis, C., Knop, M., 2006. System of centromeric, episomal, and integrative vectors based on drug resistance markers for Saccharomyces cerevisiae. Biotechniques 40, 73-78.
26 Trumbly, R., 1992. Glucose repression in the yeast Saccharomyces cerevisiae. Molecular microbiology, 6, 15-21.
27 Verduyn, C., Van Kleef, R., Frank, J., Schreuder, H., Van Dijken, J. P., Scheffers, W. A., 1985. Properties of the NAD(P)H-dependent xylose reductase from the xylose-fermenting yeast Pichia stipitis. Biochem J, 226, 669-77.
28 Werpy, T., 2004. Top Value Added Chemicals From Biomass. Volume 1-Results of Screening for Potential Candidates From Sugars and Synthesis Gas. DTIC Document.
29 Wisniak, J., Hershkowitz, M., Leibowitz, R., Stein, S., 1974. Hydrogenation of xylose to xylitol. Industrial & Engineering Chemistry Product Research and Development, 13, 75-79.

1. A method of producing xylitol, comprising:
a) providing a host cell lacking xylitol deydrogenase and xylulokinase, wherein said host cell comprises a recombinant xylose reductase, a recombinant cellodextrin transporter, and a recombinant β-glucosidase; and
b) culturing the host cell in a medium comprising cellodextrin and xylose, whereby the host cell produces xylitol from the xylose.

2. The method of claim 1, wherein the recombinant xylose reductase is XYL1 or GRE3.

3. The method of claim 1, wherein the recombinant cellodextrin transporter is a cellobiose transporter

4. The method of claim 3, wherein the cellobiose transporter is CDT-1.

5. The method of claim 1, wherein the recombinant β-glucosidase is GH1-1.

6. The method of claim 1, wherein the host cell further comprises one or more recombinant polypeptides selected from the group consisting of a glucose 6-phosphate dehydrogenase, an acetyl-CoA synthetase, an isocitrate dehydrogenase, and an acetaldehyde dehydrogenase.

7. The method of claim 6, wherein the glucose 6-phosphate dehydrogenase is ZWF1.

8. The method of claim 6, wherein the acetyl-CoA synthetase is ACS1.

9. The method of claim 6, wherein the isocitrate dehydrogenase is IDP2.

10. The method of claim 6, wherein the recombinant acetaldehyde dehydrogenase is ALD6.

11. The method of claim 1, wherein the medium comprises cellodextrin and xylose in a g/L ratio of 1:1, 1:2, or 1:3.

12. The method of claim 1, wherein the host cell is cultured at a pH of at least 5, at least 5.5, at least 6, at least 6.5, or at least 7.

13. The method of claim 1, wherein the host cell is a fungal cell.

14. The method of claim 1, wherein the host cell is a yeast cell.

15. The method of claim 14, wherein the yeast cell is S. cerevisiae.
